(19) United States
(12) Patent Application Publication
(10) Pub. No.: US 2010/0330614 A1
(43) Pub. Date: Dec. 30, 2010

(54) GLOBAL TRANSCRIPTION MACHINERY ENGINEERING TARGETING THE RNAP ALPHA SUBUNIT (RPOA)

(75) Inventors: Gregory Stephanopoulos, Winchester, MA (US); Daniel Klein-Marcuschamer, San Francisco, CA (US); Hal S. Alper, Austin, TX (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/741,750

(22) PCT Filed: Nov. 6, 2008
(86) PCT No.: PCT/US08/12511

§ 371 Date: Aug. 13, 2010

Related U.S. Application Data
(60) Provisional application No. 61/002,025, filed on Nov. 6, 2007.
(60) Provisional application No. 61/097,131, filed on Sep. 15, 2008.

(51) Int. Cl. C12P 1/00; C12N 15/63; C40B 40/08; C40B 40/02

(52) U.S. Cl. 435/41; 435/471; 506/17; 506/14

(57) ABSTRACT

The invention relates to global transcription machinery engineering to produce altered cells having improved phenotypes.

### RELATED APPLICATIONS

This application claims the benefit under 35 U.S.C. §119(e) of U.S. provisional application 61/002,025, filed Nov. 6, 2007, and U.S. provisional application 61/097,131, filed Sep. 15, 2008, the entire disclosures of which are incorporated herein by reference.

### FIELD OF THE INVENTION

The invention relates to global transcription machinery engineering to produce altered cells having improved phenotypes.

### BACKGROUND OF THE INVENTION

It is now generally accepted that many important cellular phenotypes, from disease states to metabolite overproduction, are affected by many genes. Yet, most cell and metabolic engineering approaches rely almost exclusively on the deletion or over-expression of single genes due to experimental limitations in vector construction and transformation efficiencies. These limitations preclude the simultaneous exploration of multiple gene modifications and confine gene modification searches to restricted sequential approaches where a single gene is modified at a time.

Global regulators are proteins, components of a cell's machinery, which coordinate general activities, such as transcription. The present technology suggests the engineering of these regulators to elicit complex phenotypic traits that cannot be otherwise introduced in the cells.

A critical enzyme governing the transcription in prokaryotes is RNA polymerase (RNAP). RNAP interacts with promoter DNA, in a region spanning from about 50-60 by upstream to 20 by downstream of the transcription initiation site (Record, M. T. et al. (1996) Am. Soc. Microbiol., Washington, D.C., Vol. 1, pp. 792-820; Ozoline, O. N. & Tsyganov, M. A. (1995) Nucleic Acids Res. 23, 4533-4541). Each of the four principal RNAP subunits (σ, α, β, and β′) contacts promoter DNA. The specificity subunit (sigma, a) contacts at least three promoter regions: the −10 hexamer, extended −10 region, and −35 hexamer (Record, M. T. et al. (1996)). The beta-subunits (β-, and β′) form the catalytic center of the enzyme and contact DNA in the vicinity of and downstream from the transcription-start site (Korzheva, N. & Mustaev, A. (2001) Curr. Opin. Microbiol. 4, 119-125; Murakami, K. & Darst, S. (2003) Curr. Opin. Struct. Biol. 1, 31-39; Naryshkin, N. et al. (2000) Cell 101, 601-611). Contacts of RNAP with “upstream DNA” (defined as DNA located upstream of the −35 hexamer) are mediated by the C-terminal domains of the two alpha (α)-subunits (Naryshkin, N., et al. (2000) Cell 101, 601-611; Ross, W et al. (1993) Science 262, 1407-1413; Kolb, A. et al. (1993) Nucleic Acids Res. 21, 319-326). The αCTDs bind in a sequence-specific manner at two preferred positions in the A+T-rich upstream DNA sequences referred to as UP elements (“proximal” and “distal”) Estrem, S. T. et al. (1999) Genes Dev. 13, 2134-2147). UP elements have been characterized in several bacterial species and can increase promoter activity dramatically (Kolb, A. et al. (1993); Estrem, S. T. et al. (1999); Banner, C. D. et al. (1983) J. Mol. Biol. 168, 351-365; Rao, L. et al. (1994) J. Mol. Biol. 235, 1421-1435; Fredrick, K. et al. (1995) Proc. Natl. Acad. Sci. USA 92, 2582-2586; Helmann, J. D. (1995) Nucleic Acids Res. 23, 2351-2360; Estrem, S. T. et al. (1998) Proc. Natl. Acad. Sci. USA 95, 9761-9766; Hirvonen, C. A. et al. (2001) J. Bacteriol. 183, 6305-6314). In addition to interacting specifically with UP elements, αCTD also interacts nonspecifically with upstream DNA in promoters that lack UP elements. DNA recognition by the αCTD involves a number of amino acid residues in αCTD, most notably R265.

Engineering global regulators can be a powerful tool for directed evolution introducing variability to whole organisms to generate desirable phenotypes in cells.

### SUMMARY OF THE INVENTION

The invention utilizes global transcription machinery engineering (gTME) of the alpha subunit of bacterial RNA polymerase to produce altered cells having improved phenotypes. Global transcription machinery engineering has been successfully applied for the improvement of ethanol tolerance and productivity in Saccharomyces cerevisiae (Alper et al. (2006) Science 314, 1565-68) and more recently in Escherichia coli (Alper and Stephanopoulos (2007) Metabol Eng 9, 258-67). As such, it is a promising approach for improving the industrial production of different target products by engineered microbes. In particular, the invention is demonstrated through the generation of mutated bacterial alpha subunit (RpoA). The cells resulting from introduction of the mutated alpha subunit have rapid and marked improvements in phenotypes, such as tolerance of deleterious culture conditions (e.g., solvent tolerance, exemplified by butanol) or improved production of metabolites, such as tyrosine and hyaluronic acid.

As described above, the specificity of the RNA polymerase is conferred by sigma factors and the alpha subunit, and therefore they control which set of genes is transcribed at any time (Busby S, and R. H. Ebright (1994) Cell 79, 743-46). Sigma factor engineering is reported in PCT published application WO 2007/038564, the teachings of which are incorporated by reference herein.

The alpha subunit (encoded by the gene rpoA) can modulate RNAP binding through its association with transcription activators or repressors that sit in the DNA regions far upstream of the promoter, and different mutations have been found to decrease such interactions (Ross W et al. (1993) Science 262, 1407-13). As such, the alpha subunit of the core polymerase can be thought as a regulator of global transcription.

Targeting the alpha subunit (RpoA) as a regulator of global transcription for mutation has several advantages. As mentioned above, the alpha subunit (RpoA) contributes to RNAP-DNA interaction through DNA elements, such as the UP elements, that are different from those contacted by sigma factors. The interaction of RpoA with UP elements in turn is mediated or enhanced by a variety of activator and inhibitor proteins that occupy the UP elements or DNA regions upstream of the UP elements. Unlike sigma factors, the alpha subunit is always associated with RNAP regardless of stress conditions, and the resulting enzyme is associated with promoters sets different from those covered by sigma factors. It is likely that the alpha subunit interacts with most promoters (Ross and Gourse (2005) PNAS 102, 291-96), implying a larger coverage of transcription space. In addition, each RNAP complex has two alpha subunits, and therefore two mutants could potentially synergistically alter the global transcriptome. The C-terminus of the alpha subunit (αCTD) is involved in contacting DNA at UP elements or other DNA elements while the N-terminal domain of the alpha subunit is involved in contacting RNAP. Mutations in either the N-terminal or C-terminal portion of alpha subunit may lead to different deficiencies or enhancements of the interactions governed by the two portions, potentially altering the global transcription machinery in different ways.

The introduction of mutant transcription machinery into a cell, combined with methods and concepts of directed evolution, allows one to explore a vastly expanded search space in a high throughput manner by evaluating multiple, simultaneous gene alterations in order to improve complex cellular phenotypes.

In general, engineering regulators of global transcription, such as RpoA, should impact the relative levels of message RNA and the corresponding proteins in the cell hence impacting the cellular phenotype. Therefore they are good tools for improving phenotypes that involve the activity of many gene products. This may overcome limitations encountered in classical metabolic engineering approaches, in which individual target genes are deleted or overexpressed in order to manipulate a biochemical pathway. Engineering of global regulators may simultaneously alter the fluxes of many pathways without the need of knowing the function of all the involved gene products.

The commercial applications of these technologies are diverse, because the industrial use of biocatalysts usually require improvement of complex traits. These traits include tolerance to different stresses like high or low temperature, extreme pH, or specific compounds. One application, for example, is the production of organic acids in Escherichia coli, which as been suggested as an alternative to traditional chemical synthesis from oil derivatives. These include succinic, malic, levulinic and other acids that comprise multimillion-dollar markets (Warnecke T. and R. T. Gill (2005) Microbial Cell Factories 4, 25-33). One of the main limitations of the new approach is the poor tolerance of Escherichia coli to high concentration of the products. Since tolerance to acid involves the products of many genes (proton pumps, chaperonins, amino acid synthesis and transport, etc.), global regulator engineering may find a solution. Ethanol tolerance for biofuel production is another example.

Other implications arise from optimization of classical metabolic engineering platforms. Redirection of the metabolic fluxes may increase the yields by shifting the cellular resources towards the product of interest.

Directed evolution through iterative rounds of mutagenesis and selection has been successful in broadening properties of antibodies and enzymes (W. P. Stemmer, Nature 370, 389-91 (1994)). These concepts have been recently extended and applied to non-coding, functional regions of DNA in the search for libraries of promoter activity spanning a broad dynamic range of strength as measured by different metrics (H. Alper, C. Fischer, E. Nevoigt, G. Stephanopoulos, Proc Natl Acad Sci USA 102, 12678-12683 (2005)). These evolution-inspired approaches can also be directed towards the systematic modification of the global transcription machinery as a means of improving cellular phenotype. Such modified transcription machinery units offer the opportunity to introduce simultaneous global transcription-level alterations.

According to one aspect of the invention, methods for altering the phenotype of a cell are provided, particularly involving the generation of mutated bacterial alpha subunit (RpoA). The methods comprise mutating a nucleic acid encoding ribonucleic acid polymerase (RNAP) alpha subunit RpoA and, optionally, its promoter, expressing the nucleic acid in a prokaryotic cell to provide an altered cell that includes the mutated nucleic acid encoding RpoA, and culturing the altered cell. In some embodiments, the methods also include determining the phenotype of the altered cell or comparing the phenotype of the altered cell with the phenotype of the cell prior to alteration. In further embodiments, the methods also include mutating additional nucleic acids encoding global transcription machinery, other than RpoA. In preferred embodiments, the nucleic acids encoding global transcription machinery is a rpoD (σ⁷⁰) gene, a rpoF (σ²⁸) gene, a rpoS (σ³⁸) gene, a rpoH (σ³²) gene, a rpoN (σ⁵⁴) gene, a rpoE (σ²⁴) gene or a fed (σ¹⁹) gene.

In other embodiments, the methods also include repeating the mutation of the nucleic acid to produce a n^{th} generation altered cell. In still other embodiments, the methods also include determining the phenotype of the n^{th} generation altered cell or comparing the phenotype of the n^{th} generation altered cell with the phenotype of any prior generation altered cell or of the cell prior to alteration. In preferred embodiments, the step of repeating the mutation of the nucleic acid encoding RpoA comprises isolating a nucleic acid encoding the mutated nucleic acid encoding RpoA and optionally, its promoter, from the altered cell, mutating the nucleic acid, and introducing the mutated nucleic acid into another cell.

In certain embodiments, the cell is a prokaryotic cell, preferably a bacterial cell or an archaeal cell. In some embodiments the nucleic acid encoding the RNAP alpha subunit RpoA is part of an expression vector. In some embodiments the RNAP alpha subunit RpoA is expressed from an expression vector.

The nucleic acid in certain embodiments is a member of a collection (e.g., a library) of nucleic acids. Thus the methods of the invention include, in some embodiments, introducing the collection into the cell.

In further embodiments, the step of expressing the nucleic acid includes integrating the nucleic acid into the genome or replacing a nucleic acid that encodes the endogenous RpoA.

The mutation of the nucleic acid, in certain embodiments, includes directed evolution of the nucleic acid, such as mutation by error prone PCR or mutation by gene shuffling. In other embodiments, the mutation of the nucleic acid includes synthesizing the nucleic acid with one or more mutations. Nucleic acid mutations in the invention can include one or more point mutations, and/or one or more truncations and/or deletions.

In some embodiments of the invention, the DNA binding region of the RNAP alpha subunit RpoA is not disrupted or removed by the one or more truncations or deletions. In other embodiments, a promoter upstream element (UP element) binding region of the RNAP alpha subunit RpoA is not disrupted or removed by the one or more truncations or deletions. In yet other embodiments a carboxy-terminal portion of the RNAP alpha subunit RpoA is not disrupted or removed by the one or more truncations or deletions. In another embodiment an amino-terminal portion of the RNAP alpha subunit RpoA is not disrupted or removed by the one or more truncations or deletions.

In certain embodiments the mutated nucleic acid encoding RpoA exhibits increased transcription of genes relative to the unmutated nucleic acid encoding RpoA, decreased transcription of genes relative to the unmutated nucleic acid encoding RpoA, increased repression of gene transcription relative to the unmutated nucleic acid encoding RpoA, and/or decreased repression of gene transcription relative to the unmutated nucleic acid encoding RpoA

In still other embodiments, the methods also include selecting the altered cell for a predetermined phenotype. Preferably, the step of selecting includes culturing the altered cell under selective conditions and/or high-throughput assays of individual cells for the phenotype.

A wide variety of phenotypes can be selected in accordance with the invention. In some preferred embodiments, the phenotype is increased tolerance of deleterious culture conditions. Such phenotypes include: solvent tolerance or hazardous waste tolerance, e.g., butanol, propane, ethanol, hexane or cyclohexane; tolerance of industrial media; tolerance of high sugar concentration; tolerance of high salt concentration; tolerance of butyrate, tolerance of high temperatures; tolerance of extreme pH; tolerance of surfactants, tolerance of osmotic stress and tolerance of a plurality of deleterious conditions, such as for example tolerance of high sugar and ethanol concentrations, butyrate and butanol concentrations, or butyrate and propane concentrations.

In other preferred embodiments, the phenotype is increased metabolite production. Metabolites include L-tyrosine, lycopene, ethanol, polyhydroxybutyrate (PHB), and therapeutic proteins, such as an antibody or an antibody fragment.

In still other preferred embodiments, the phenotype is tolerance to a toxic substrate, metabolic intermediate or product. Toxic metabolites include organic solvents, acetate, para-hydroxybenzoic acid (pHBA), hyaluronic acid and overexpressed proteins. In yet other embodiments, the phenotype is antibiotic resistance.

The cell used in the methods can be optimized for the phenotype prior to mutating the nucleic acid encoding RpoA.

The methods of the invention, in certain embodiments, also include identifying the changes in gene expression in the altered cell. The changes in gene expression preferably are determined using a nucleic acid microarray.

According to another aspect of the invention, methods for altering the phenotype of a cell are provided. The methods include altering the expression of one or more gene products in a first cell that are identified by detecting changes in gene expression in a second cell, wherein the changes in gene expression in the second cell are produced by mutating a nucleic acid encoding ribonucleic acid polymerase (RNAP) alpha subunit RpoA of the second cell. In some embodiments, altering the expression of the one or more gene products in the first cell includes increasing expression of one or more gene products that were increased in the second cell. In some preferred embodiments, the expression of the one or more gene products is increased by introducing into the first cell one or more expression vectors that express the one or more gene products, or by increasing the transcription of one or more endogenous genes that encode the one or more gene products. In the latter embodiments, increasing the transcription of the one or more endogenous genes includes mutating a transcriptional control (e.g., promoter/enhancer) sequence of the one or more genes. In other embodiments, altering the expression of the one or more gene products in the first cell includes decreasing expression of one or more gene products that were decreased in the altered cell. Preferably, the expression of the one or more gene products is decreased by introducing into the first cell nucleic acid molecules that reduce the expression of the one or more gene products, such as nucleic acid molecules that are, or express, siRNA molecules. In other embodiments, the expression of the one or more gene products is decreased by mutating one or more genes that encode the one or more gene products or a transcriptional control (e.g., promoter/enhancer) sequence of the one or more genes.

The changes in gene expression in the second cell preferably are determined using a nucleic acid microarray.

In other embodiments, the changes in gene expression in the second cell are used to construct a model of a gene or protein network, and the model is used to select which of the one or more gene products in the network to alter.

Also provided according to the invention are cells produced by the foregoing methods.

According to another aspect of the invention, methods for altering the production of a metabolite are provided. The methods include mutating, according to any of the foregoing methods, ribonucleic acid polymerase (RNAP) alpha subunit RpoA of a prokaryotic cell that produces a selected metabolite to produce an altered cell, and isolating altered cells that produce increased or decreased amounts of the selected metabolite. In some embodiments, the methods also include culturing the isolated cells, and recovering the metabolite from the cells or the cell culture. Preferred metabolites include L-tyrosine, lycopene, ethanol, polyhydroxybutyrate (PHB), hyaluronic acid, and therapeutic proteins, such as recombinant proteins, antibodies or antibody fragments.

In some embodiments the cells are prokaryotic cells, including bacterial cells or archaeal cells.

According to another aspect of the invention, collections (e.g., a library) including a plurality of different nucleic acid molecule species are provided, in which it is preferred that each nucleic acid molecule species encodes ribonucleic acid polymerase (RNAP) alpha subunit RpoA comprising different mutation(s). In certain embodiments, the collection includes additional nucleic acid molecule species encoding sigma factors, such as the rpoD (σ⁷⁰) gene, the rpoF (σ²⁸) gene, the rpoS (σ³⁸) gene, the rpoH (σ³²) gene, the rpoN (σ⁵⁴) gene, the rpoE (σ²⁴) gene or the feel (σ¹⁹) gene.

In certain embodiments, the nucleic acid molecule species are contained in expression vectors. The expression vectors preferably contain a plurality of different nucleic acid molecule species, wherein each nucleic acid molecule species encodes different RNAP alpha subunit RpoA mutations.

In other embodiments, the nucleic acid encoding RpoA is mutated by directed evolution, which preferably is performed using error prone PCR and/or using gene shuffling. Preferred mutation(s) in the RNAP alpha subunit RpoA is/are one or more point mutations and/or one or more truncations and/or deletions. In some embodiments, the truncation does not include the DNA binding region of the RNAP alpha subunit RpoA. In other embodiments, the truncation does not include the UP element binding region of the RNAP alpha subunit RpoA. In still other embodiments, the truncation does not include the carboxy-terminal portion of the RNAP alpha subunit RpoA. n yet other embodiments the truncation does not include amino-terminal portion of the RNAP alpha subunit RpoA. In still other embodiments, the RNAP alpha subunit RpoA of a cell is mutated according to any of the foregoing methods.

In a further aspect of the invention, collections (e.g., a library) of cells is provide that includes the foregoing collections of nucleic acid molecules. In some embodiments, the collection includes a plurality of cells, each of the plurality of cells comprising one or more of the nucleic acid molecules. The cells preferably are prokaryotic cells, such as bacterial cells or archaeal cells. In other embodiments, the nucleic acid molecules are integrated into the genome of the cells or replace nucleic acids that encode the endogenous RNAP alpha subunit RpoA.

According to still another aspect of the invention, nucleic acid encoding ribonucleic acid polymerase (RNAP) alpha subunit RpoA produced by a plurality of rounds of mutation are provided. The plurality of rounds of mutation preferably include directed evolution, such as that performed by mutation by error prone PCR and/or mutation by gene shuffling. In some embodiments, the nucleic acid encodes a plurality of different RNAP alpha subunit RpoA mutations. The nucleic acid preferably encodes a plurality of different versions of the same type of RNAP alpha subunit RpoA species.

Also provided according to the invention is ribonucleic acid polymerase (RNAP) alpha subunit RpoA encoded by the foregoing nucleic acids.

According to a further aspect of the invention, methods for bioremediation of a selected waste product are provided. The methods include mutating, according to any of the foregoing methods, RNAP alpha subunit RpoA of a prokaryotic cell to produce an altered cell, isolating altered cells that metabolize an increased amount of the selected waste product relative to unaltered cells, culturing the isolated cells, and exposing the altered cells to the selected waste product, thereby providing bioremediation of the selected waste product.

According to another aspects of the invention, methods for identifying a cell that produces mucopolysaccharides are provided. The methods include adding Alcian blue solution to media, in which cells suspected of being mucopolysaccharide-producing cells were cultured, to obtain a mixture; heating and subsequently cooling the mixture, separating the soluble and insoluble fractions of the mixture from, measuring optical density (OD) of the soluble fraction, and comparing the value obtained for the measurement to a standard to obtain a concentration value. A concentration value higher than 0 indicates that the cell being identified produces mucopolysaccharides.

In some embodiments, the methods for identifying a cell that produces mucopolysaccharides include adding Alcian blue solution to media containing mucopolysaccharide-producing cells and obtaining a mixture, heating and subsequently cooling the mixture, separating the soluble and insoluble fractions of the mixture, measuring optical density (OD) of the soluble fraction to obtain a value, and comparing the obtained value with a control. A value obtained being higher than that of the control being indicative that the cell produces mucopolysaccharides.

In preferred embodiments the mucopolysaccharide-producing cells produce hyaluronic acid. Cells useful for the aforementioned methods are prokaryotic cells, preferably bacterial cells or archaeal cells. In some embodiments the bacterial cell is Gram-negative. In other embodiments the prokaryotic cell is Streptococcus or Bacillus subtilis.

According to a yet another aspect of the invention, methods for identifying a recombinant bacterial cell that produces hyaluronic acid (HA) are provided. The methods include plating bacteria on solid medium supplemented with sorbitol, incubating the bacteria to form colonies, and identifying as HA-producing bacterial cells those colonies that are translucent. In preferred embodiments, the solid medium is LB medium supplemented with sorbitol, Magnesium Chloride, ampicillin and L-arabinose (LBSMA), and further supplemented with a second antibiotic. In some embodiments identification of a colony as translucent is performed by visually comparing translucency of the colonies with colonies from cells not producing HA. In other embodiments the degree of translucency of colonies from cells that produce HA as compared to colonies from cells not producing HA is being correlated with the amount of HA being produced by the cell. In preferred embodiments, the recombinant bacterial cell is an Escherichia coli cell. In yet other embodiments the aforementioned methods are employed in a high-throughput screen identifying a cell that produces mucopolysaccharides in a collection of cells carrying different mutations, including mutations in RNAP alpha subunit RpoA or sigma factor.

In some embodiments methods for altering the phenotype of a cell involve mutating the alpha CTD domain of RNAP. In some embodiments the mutation in RNAP is a substitution of amino acid 299, optionally from a serine residue to a threonine residue. A cell containing a mutated form of the alpha CTD domain of RNAP can be cultured in the presence of butyrate, resulting in isolation of a cell that has an increased growth rate in the presence of butyrate relative to a wildtype cell. Culturing of cells that are tolerant to butyrate can be used to produce and collect butanol and/or propane.

Aspects of the invention relate to methods for producing a cell that is tolerant to butyrate, involving: mutating the alpha CTD domain of a nucleic acid encoding ribonucleic acid polymerase (RNAP) alpha subunit RpoA, expressing the nucleic acid in a cell to provide an altered cell that includes the mutated nucleic acid encoding RpoA, culturing the cell in butyrate, and isolating a cell that is tolerant to butyrate. In some embodiments the mutation in RNAP is a substitution of amino acid 299, optionally from a serine residue to a threonine residue. Such methods can be used to isolate a cell that has an increased growth rate in the presence of butyrate relative to a wildtype cell. Such a cell can be cultured and used to produce and collect butanol/or propane.

Aspects of the invention relate to methods for increasing the growth rate of a cell that contains recombinant global transcription machinery involving expressing the recombinant global transcription machinery using a strong promoter. In some embodiments the recombinant global transcription machinery is mutated. In some embodiments the recombinant global transcription machinery is RpoA. In some embodiments the promoter is P_{spc}.

Aspects of the invention relate to methods for optimizing a cellular library. In some embodiments the method involves: applying localized mutagenesis to the library, and calculating the level of phenotypic diversity, wherein the rate of mutagenesis is optimized to achieve maximum phenotypic diversity.

These and other aspects of the invention, as well as various embodiments thereof, will become more apparent in reference to the drawings and detailed description of the invention.

### BRIEF DESCRIPTION OF THE DRAWINGS

FIG. 1: A photographic depiction of translucent colony morphology of HA-producing recombinant E. coli and dense colony morphology of non-HA producing recombinant E. coli. Translucent colonies are marked by dashed arrows, and dense colonies are marked by solid arrows.

FIG. 2: Graphs depicting absorbance spectra of pure alcian blue solution and alcian blue mixed with HA. (a), scanned spectrum of 10 μl alcian blue solution in 990 μl 3% acetic acid buffer, using the buffer as blank control; (b, c and d), negative absorbance of 10, 50 and 100 μl, respectively, alcian blue and HA solution in a total volume of 1 ml using the corresponding alcian blue solution without HA as blank control. The scanned samples were prepared as follows: 10, 50 and 100 μl alcian blue solution were mixed with 500 μl of 400 mg/L HA and 3% acetic acid buffer was added to 1 mL, the mixture was microwaved 30 seconds, cooled for 1 h at room temperature and centrifuged 1 min at 10000 rpm. The supernatant was loaded into the UV-cuvette, and the spectrum scanned from 200-800 nm. Optimal absorbance peaks: (a), 334 nm and 605 nm, positive; (b), 334 nm and 605 nm, negative; (c), 380, 560 and 700 nm, negative; (d), 400, 540 and 730 nm, negative. Experiments repeated 3 times.

FIG. 3: Bar graphs depicting an intensity comparison of the HA-stained alcian blue solution.

FIG. 4: Diagram depicting HA quantification by alcian blue staining. Different standing time for HA and alcian blue binding at room temperature was evaluated (30 min, 1, 2, 5 and 5.0 h) within the HA concentration range of 0-500 mg/L.

FIG. 5: Diagram depicting a standard curve of alcian blue staining for HA quantification. A second-order polynomial fit was used. HA (mg/L)=926.33818-2077.15527*OD₅₄₀+1228.74084*OD₅₄₀², R²=0.99945. (Insert) the 5-wells of alcian blue solution added with different concentrations of HA. The binding time of the alcian blue and HA was 2.5 h.

FIG. 6: Bar graphs depicting a library screening of optimal E. coli for HA accumulation using alcian blue quantification. Control strain, Top10/pMBAD-sseABC. The details for the library screening were stated in Materials and Methods. All samples were measured in duplicate.

FIG. 7: Bar graphs depicting tyrosine production (mg/ml) by two rpoA mutants strains—rpoA14 and rpoA27—that were generated by transforming pHACm-rpoA plasmid libraries into E. coli K12 ΔpheA tyrR::P_{LtetO-1}tyrA^{fbr}aroG^{fbr}lacZ::P_{LtetO-1}tyrA^{fbr}aroG^{fbr} parental strain, and isolated after screening with a melanin-based assay.

FIG. 8: Graphs depicting (A) the change of pH and (B) the change of acetate production (mg/l) in medium over time when culturing rpoA mutants strains rpoA14 and rpoA27 or rpoA-wt parental strains.

FIG. 9: Bar graphs depicting overnight growth of DH5α cells transformed with either the wild-type or the L33 mutant of rpoA in different alcohol solvents, measured as cell density (Mao). The abbreviations are: 1-C4 for n-butanol, 2-C4 for isobutanol, 1-05 for n-pentanol, and 3-05 for 3-pentanol. The concentration used is in parenthesis (v/v).

FIG. 10: Bar graph depicting divergence in various rpoA mutant libraries. The divergence is a statistical measure that describes the additional phenotypic distance of the libraries compared to that of the wild-type and was calculated as described (Klein-Marcuschamer et al., Proc Natl Acad Sci USA 105:2319-24, 2008). It uses intracellular pH as the phenotype both in growing and non-growing cells. The divergence value is a relative measure and has no strict physical meaning; it is used only for comparing different populations. Libraries are named following the nomenclature of Example 5.

FIG. 11: Bar graph depicting enrichment of improved clones. The graph shows the maximum recorded advantage in OD (600 nm) of cultures of the libraries relative to the control in different screening conditions, that is, the theoretical enrichment of improved clones. The conditions are: 1) M9 medium, 15 g/L butyrate throughout screening; 2) MOPS medium supplemented with amino acids (5%), decreasing butyrate concentration (18, 15, 12 g/L); 3) MOPS medium, 15 g/L butyrate throughout screening; 4) MOPS medium supplemented with amino acids, 15 g/L butyrate throughout screening. For αCTD*L, two repeats of the last set of conditions are given by runs αCTD*L 5 and 6. For rpoA*L, rpoA*M, and rpoA*H, some conditions were tried more than once (not shown) to rule out experimental error as the reason for not obtaining improved mutants. Even though a positive theoretical enrichment is shown, no improved mutant was isolated in any library except the αCTD*L, suggesting that transient advantages of up to ˜15% can be considered noise.

FIG. 12: Bar graph depicting growth rates of K12 recA⁻ transformed with wild-type or mutant versions of rpoA under two promoters (lac and spc). Mutants #16 and #1 have the same amino acid sequence, but an additional synonymous mutation in #16 changes a common codon for glycine to a more uncommon one. P_{lac} is the left bar in each set of bars; P_{spc} is the right bar in each set of bars. As shown, increasing the expression level of the mutant (using P_{spc}, right bar in each set) increases the growth advantage over the wild-type by up to 60%.

FIG. 13: Flow chart for guiding strain improvement using mutant libraries. Nomenclature: i, number of libraries constructed; y, number of screening experiments; T, total budget available (in money or time); B, cost of building a new library; S, cost of screening a library; Pᵢ relative probability of success of library i, as quantified by phenotypic diversity; Pₘₐₓ, maximum phenotypic diversity available.

### DETAILED DESCRIPTION OF THE INVENTION

Global transcription machinery is responsible for controlling the transcriptome in all cellular systems (prokaryotic and eukaryotic). In bacterial systems, the alpha subunit RpoA and the sigma factors play a critical role in orchestrating global transcription by focusing the promoter preferences of the RNA polymerase holoenzyme, RNAP (R. R. Burgess, L. Anthony, Curr. Opin. Microbiol. 4, 126-131 (2001)).

Traditional strain improvement paradigms rely predominantly on making sequential, single-gene modifications and often fail to reach the global maxima. The reason is that metabolic landscapes are complex (H. Alper, K. Miyaoku, G. Stephanopoulos, Nat Biotechnol 23, 612-616 (2005); H. Alper, Y.-S. Jin, J. F. Moxley, G. Stephanopoulos, Metab Eng 7, 155-164 (2005)) and incremental or greedy search algorithms fail to uncover synthetic mutants that are beneficial only when all mutations are simultaneously introduced. Protein engineering on the other hand can quickly improve fitness, through randomized mutagenesis and selection for enhanced antibody affinity, enzyme specificity, or catalytic activity (E. T. Boder, K. S. Midelfort, K. D. Wittrup, Proc Natl Acad Sci USA 97, 10701-5 (2000); A. Glieder, E. T. Farinas, F. H. Arnold, Nat Biotechnol 20, 1135-9 (2002); N. Varadarajan, J. Gam, M. J. Olsen, G. Georgiou, B. L. Iverson, Proc Natl Acad Sci USA 102, 6855-60 (2005)). An important reason for the drastic enhancement obtained in these examples is the ability of these methods to probe a significant subset of the huge amino acid combinatorial space by evaluating many simultaneous mutations. In this invention, we exploit the global regulatory functions of the RNAP alpha subunit (RpoA) to similarly introduce multiple simultaneous gene expression changes and thus facilitate whole-cell engineering by selecting mutants responsible for improved cellular phenotype.

The invention provides methods for altering the phenotype of a cell. In the methods include mutating a nucleic acid encoding a global transcription machinery protein and, optionally, its promoter, expressing the nucleic acid in a cell to provide an altered cell that includes a mutated global transcription machinery protein, and culturing the altered cell. As used herein, “global transcription machinery” is one or more molecules that modulates the transcription of a plurality of genes. The global transcription machinery can be proteins that affect gene transcription by interacting with and modulating the activity of a RNA polymerase molecule, such as the RNAP alpha subunit (RpoA), encoded by the gene rpoA, as well as for example sigma factors encoded by the genes rpoD (σ⁷⁰), rpoF (σ²⁸), rpoS (σ³⁸), rpoH (σ³²), rpoN (σ⁵⁴), rpoE (σ²⁴) and fecI (σ¹⁹). The global transcription machinery also can be proteins that alter the ability of the genome of a cell to be transcribed (e.g., methyltransferases, histone methyltransferases, histone acetylases and deacetylases). Further, global transcription machinery can be molecules other than proteins (e.g., micro RNAs) that alter transcription of a plurality of genes. Global transcription machinery particularly useful in accordance with the invention include bacterial RNAP alpha subunit (RpoA) and sigma factors.

In many instances, the process of mutating the global transcription machinery will include iteratively making a plurality of mutations of the global transcription machinery, but it need not, as even a single mutation of the global transcription machinery can result in dramatic alteration of phenotype, as is demonstrated herein.

While the methods of the invention typically are carried out by mutating the global transcription machinery followed by introducing the mutated global transcription machinery into a cell to create an altered cell, it is also possible to mutate endogenous global transcription machinery genes, e.g., by replacement with mutant global transcription machinery or by in situ mutation of the endogenous global transcription machinery. As used herein, “endogenous” means native to the cell; in the case of mutating global transcription machinery, endogenous refers to the gene or genes of the global transcription machinery that are in the cell. In contrast, the more typical methodology includes mutation of a global transcription machinery gene or genes outside of the cell, followed by introduction of the mutated gene(s) into the cell.

Using standard recombinant genetic techniques, the global transcription machinery genes, e.g. the rpoA gene, encoding the RNAP alpha subunit, can be mutated in the same prokaryotic species or bacterial strain or different prokaryotic species or bacterial strain as the cell into which they are introduced.

Alternatively, global transcription machinery from different prokaryotic species or bacterial strain can be utilized to provide additional variation in the transcriptional control of genes. For example, global transcription machinery of a Streptomyces bacterium could be mutated and introduced into E. coli. The different global transcription machinery also could be sourced from different kingdoms or phyla of organisms. Depending on the method of mutation used, same and different global transcription machinery can be combined for use in the methods of the invention, e.g., by gene shuffling.

Optionally, the transcriptional control sequences of global transcription machinery can be mutated, rather than the coding sequence itself. Transcriptional control sequences include promoter and enhancer sequences. The mutated promoter and/or enhancer sequences, linked to the global transcription machinery coding sequence, can then be introduced into the cell.

After the mutant global transcription machinery is introduced into the cell to make an altered cell, then the phenotype of the altered cell is determined/assayed. This can be done by selecting altered cells for the presence (or absence) of a particular phenotype. Examples of phenotypes are described in greater detail below. The phenotype also can be determined by comparing the phenotype of the altered cell with the phenotype of the cell prior to alteration.

In preferred embodiments, the mutation of the global transcription machinery and introduction of the mutated global transcription machinery are repeated one or more times to produce an “n^{th} generation” altered cell, where “n” is the number of iterations of the mutation and introduction of the global transcription machinery. For example, repeating the mutation and introduction of the global transcription machinery once (after the initial mutation and introduction of the global transcription machinery) results in a second generation altered cell. The next iteration results in a third generation altered cell, and so on. The phenotypes of the cells containing iteratively mutated global transcription machinery then are determined (or compared with a cell containing non-mutated global transcription machinery or a previous iteration of the mutant global transcription machinery) as described elsewhere herein.

The process of iteratively mutating the global transcription machinery allows for improvement of phenotype over sequential mutation steps, each of which may result in multiple mutations of the global transcription machinery. It is also possible that the iterative mutation may result in mutations of particular amino acid residues “appearing” and “disappearing” in the global transcription machinery over the iterative process.

In a typical use of the methodology, the global transcription machinery is subjected to directed evolution by mutating a nucleic acid molecule that encodes the global transcription machinery. A preferred method to mutate the nucleic acid molecule is to subject the coding sequence to mutagenesis, and then to insert the nucleic acid molecule into a vector (e.g., a plasmid). This process may be inverted if desired, i.e., first insert the nucleic acid molecule into a vector, and then subject the sequence to mutagenesis, although it is preferred to mutate the coding sequence prior to inserting it in a vector.

When the directed evolution of the global transcription machinery is repeated, i.e., in the iterative processes of the invention, a preferred method includes the isolation of a nucleic acid encoding the mutated global transcription machinery and optionally, its promoter, from the altered cell. The isolated nucleic acid molecule is then mutated (producing a nucleic acid encoding a second generation mutated global transcription machinery), and subsequently introduced into another cell.

The isolated nucleic acid molecule when mutated, forms a collection of mutated nucleic acid molecules that have different mutations or sets of mutations. For example, the nucleic acid molecule when mutated randomly can have set of mutations that includes mutations at one or more positions along the length of the nucleic acid molecule. Thus, a first member of the set may have one mutation at nucleotide n1 (wherein nx represents a number of the nucleotide sequence of the nucleic acid molecule, with x being the position of the nucleotide from the first to the last nucleotide of the molecule). A second member of the set may have one mutation at nucleotide n2. A third member of the set may have two mutations at nucleotides n1 and n3. A fourth member of the set may have two mutations at positions n4 and n5. A fifth member of the set may have three mutations: two point mutations at nucleotides n4 and n5, and a deletion of nucleotides n6-n7. A sixth member of the set may have point mutations at nucleotides n1, n5 and n8, and a truncation of the 3′ terminal nucleotides. A seventh member of the set may have nucleotides n9-n10 switched with nucleotides n11-n12. Various other combinations can be readily envisioned by one of ordinary skill in the art, including combinations of random and directed mutations.

The collection of nucleic acid molecules can be a library of nucleic acids, such as a number of different mutated nucleic acid molecules inserted in a vector. Such a library can be stored, replicated, aliquoted and/or introduced into cells to produce altered cells in accordance with standard methods of molecular biology.

Mutation of the global transcription machinery for directed evolution preferably is random. However, it also is possible to limit the randomness of the mutations introduced into the global transcription machinery, to make a non-random or partially random mutation to the global transcription machinery, or some combination of these mutations. For example, for a partially random mutation, the mutation(s) may be confined to a certain portion of the nucleic acid molecule encoding the global transcription machinery.

The method of mutation can be selected based on the type of mutations that are desired. For example, for random mutations, methods such as error-prone PCR amplification of the nucleic acid molecule can be used. Site-directed mutagenesis can be used to introduce specific mutations at specific nucleotides of the nucleic acid molecule. Synthesis of the nucleic acid molecules can be used to introduce specific mutations and/or random mutations, the latter at one or more specific nucleotides, or across the entire length of the nucleic acid molecule. Methods for synthesis of nucleic acids are well known in the art (e.g., Tian et al., Nature 432: 1050-1053 (2004)).

DNA shuffling (also known as gene shuffling) can be used to introduce still other mutations by switching segments of nucleic acid molecules. See, e.g., U.S. Pat. No. 6,518,065, related patents, and references cited therein. The nucleic acid molecules used as the source material to be shuffled can be nucleic acid molecule(s) that encode(s) a single type of global transcription machinery (e.g., RNAP alpha subunit RpoA), or more than one type of global transcription machinery.

A variety of other methods of mutating nucleic acid molecules, in a random or non-random fashion, are well known to one of ordinary skill in the art. One or more different methods can be used combinatorially to make mutations in nucleic acid molecules encoding global transcription machinery. In this aspect, “combinatorially” means that different types of mutations are combined in a single nucleic acid molecule, and assorted in a set of nucleic acid molecules. Different types of mutations include point mutations, truncations of nucleotides, deletions of nucleotides, additions of nucleotides, substitutions of nucleotides, and shuffling (e.g., re-assortment) of segments of nucleotides. Thus, any single nucleic acid molecule can have one or more types of mutations, and these can be randomly or non-randomly assorted in a set of nucleic acid molecules. For example, a set of nucleic acid molecules can have a mutation common to each nucleic acid molecule in the set, and a variable number of mutations that are not common to each nucleic acid molecule in the set. The common mutation, for example, may be one that is found to be advantageous to a desired altered phenotype of the cell.

Preferably a promoter binding region of the global transcription machinery is not disrupted or removed by the one or more truncations or deletions.

The mutated global transcription machinery can exhibit increased or decreased transcription of genes relative to the unmutated global transcription machinery. In addition, the mutated global transcription machinery can exhibit increased or decreased repression of transcription of genes relative to the unmutated global transcription machinery.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to: plasmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a CT antigen polypeptide or fragment or variant thereof. That heterologous DNA (RNA) is placed under operable control of transcriptional elements to permit the expression of the heterologous DNA in the host cell.

When the nucleic acid molecule that encodes mutated global transcription machinery is expressed in a cell, a variety of transcription control sequences (e.g., promoter/enhancer sequences) can be used to direct expression of the global transcription machinery. The promoter can be a native promoter, i.e., the promoter of the global transcription machinery gene, which provides normal regulation of expression of the global transcription machinery. A variety of conditional promoters also can be used, such as promoters controlled by the presence or absence of a molecule, such as the tetracycline-responsive promoter (M. Gossen and H. Bujard, Proc. Natl. Acad. Sci. USA, 89, 5547-5551 (1992)).

A nucleic acid molecule that encodes mutated global transcription machinery can be introduced into a cell or cells using methods and techniques that are standard in the art, e.g. bacterial transformation by chemical or electroporation methods. Expressing the nucleic acid molecule encoding mutated global transcription machinery also may be accomplished by integrating the nucleic acid molecule into the genome or by replacing a nucleic acid sequence that encodes the endogenous global transcription machinery.

By mutating global transcription machinery, novel compositions are provided, including nucleic acid molecules encoding global transcription machinery produced by a plurality of rounds of mutation. The plurality of rounds of mutation can include directed evolution, in which each round of mutation is followed by a selection process to select the mutated global transcription machinery that confer a desired phenotype. The methods of mutation and selection of the mutated global transcription machinery are as described elsewhere herein. Global transcription machinery produced by these nucleic acid molecules also are provided.

In certain cases, it has been found that mutated global transcription machinery are truncated forms of the unmutated global transcription machinery.

The cells useful in the invention include prokaryotic cells such as bacterial cells and archaeal cells.

Examples of bacteria include Escherichia spp., Streptomyces spp., Zymonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp. and Pantoea spp.

Examples of archaea (also known as archaebacteria) include Methylomonas spp., Sulfolobus spp., Methylobacterium spp. Halobacterium spp., Methanobacterium spp., Methanococci spp., Methanopyri spp., Archaeoglobus spp., Ferroglobus spp., Thermoplasmata spp. and Thermococci spp.

Directed evolution of global transcription machinery produces altered cells, some of which have altered phenotypes. Thus the invention also includes selecting altered cells for a predetermined phenotype or phenotypes. Selecting for a predetermined phenotype can be accomplished by culturing the altered cells under selective conditions. Selecting for a predetermined phenotype also can be accomplished by high-throughput assays of individual cells for the phenotype. For example, cells can be selected for tolerance to deleterious conditions and/or for increased production of metabolites. Tolerance phenotypes include tolerance of solvents such as ethanol, and organic solvents such as hexane or cyclohexane; tolerance of toxic metabolites such as acetate, para-hydroxybenzoic acid (pHBA), para-hydroxycinnamic acid, hydroxypropionaldehyde, overexpressed proteins, organic solvents and immuno-suppressant molecules; tolerance of surfactants; tolerance of osmotic stress; tolerance of high sugar concentrations; tolerance of high temperatures; tolerance of extreme pH conditions (high or low); resistance to apoptosis; tolerance of toxic substrates such as hazardous waste; tolerance of industrial media; increased antibiotic resistance, etc. Selection for solvent tolerance, hyaluronic acid tolerance, and selection for increased production of tyrosine of are exemplified in the working examples. Hyaluronic acid (Hyaluronan, HA) is a valuable functional biopolymer, its importance stemming from its structural, rheological, physiological, and biological properties, leading to a wide range of applications in the health, cosmetic and clinical fields (Goa K L. and Benfield P. (1994) Drugs 47, 536-66; Lauren T C (1998) Portland Press Ltd, London).

As used herein with respect to altered cells containing mutated global transcription machinery, “tolerance” means that an altered cell is able to withstand the deleterious conditions to a greater extent than an unaltered cell, or a previously altered cell. For example, the unaltered or previously altered cell is a “parent” of the “child” altered cell, or the unaltered or previously altered cell is the (n−1)^{th} generation as compared to the cell being tested, which is n^{th} generation. “Withstanding the deleterious conditions” means that the altered cell has increased growth and/or survival relative to the unaltered or previously altered cell. This concept also includes increased production of metabolites that are toxic to cells.

With respect to tolerance of high sugar concentrations, such concentrations can be ≧100 g/L, ≧120 g/L, ≧140 g/L, ≧160 g/L, ≧180 g/L, ≧200 g/L, ≧250 g/L, ≧300 g/L, ≧350 g/L, ≧400 g/L, ≧450 g/L, ≧500 g/L, etc. With respect to tolerance of high salt concentrations, such concentrations can be ≧1 M, ≧2 M, ≧3 M, ≧4 M, ≧5 M, etc. With respect to tolerance of high temperatures, the temperatures can be, e.g., ≧42° C., ≧44° C., ≧46° C., ≧48° C., ≧50° C. for bacterial cells. Other temperature cutoffs may be selected according to the cell type used. With respect to tolerance of extreme pH, exemplary pH cutoffs are, e.g., ≧pH10, ≧pH11, ≧pH12, ≧pH13, or ≦pH4.0, ≦pH3.0, ≦pH2.0, ≦pH1.0. With respect to tolerance of surfactants, exemplary surfactant concentrations are ≧5% w/v, ≧6% w/v, ≧7% w/v, ≧8% w/v, ≧9% w/v, ≧10% w/v, ≧12% w/v, ≧15% w/v, etc. With respect to tolerance of ethanol, exemplary ethanol concentrations are ≧4% v/v, ≧5% v/v, ≧6% v/v, ≧7% v/v, ≧8% v/v, ≧9% v/v, ≧10% v/v, etc. With respect to tolerance of osmotic stress, exemplary concentrations (e.g., of LiCl) that induce osmotic stress are ≧100 mM, ≧150 mM, ≧200 mM, ≧250 mM, ≧300 mM, ≧350 mM, ≧400 mM, etc.

The invention includes obtaining increased production of metabolites by cells. As used herein, a “metabolite” is any molecule that is made or can be made in a cell. Metabolites include metabolic intermediates or end products, any of which may be toxic to the cell, in which case the increased production may involve tolerance of the toxic metabolite. Thus metabolites include small molecules, peptides, large proteins, lipids, sugars, etc.

The invention also provides for selecting for a plurality of phenotypes, such as tolerance of a plurality of deleterious conditions, increased production of a plurality of metabolites, or a combination of these.

It may be advantageous to use cells that are previously optimized for the predetermined phenotype prior to introducing mutated global transcription machinery.

Via the actions of the mutated global transcription machinery, the altered cells will have altered expression of genes. The methods of the invention can, in certain aspects, include identifying the changes in gene expression in the altered cell. Changes in gene expression can be identified using a variety of methods well known in the art. Preferably the changes in gene expression are determined using a nucleic acid microarray.

In some aspects of the invention, one or more of the changes in gene expression that are produced in a cell by mutated global transcription machinery can be reproduced in another cell in order to produce the same (or a similar) phenotype. The changes in gene expression produced by the mutated global transcription machinery can be identified as described above. Individual gene(s) can then be targeted for modulation, through recombinant gene expression or other means. For example, mutated global transcription machinery may produce increases in the expression of genes A, B, C, D, and E, and decreases in the expression of genes F, G, and H. The invention includes modulating the expression of one or more of these genes in order to reproduce the phenotype that is produced by the mutated global transcription machinery. To reproduce the predetermined phenotype, one or more of genes A, B, C, D, E, F, G, and H can be increased, e.g., by introducing into the cell expression vector(s) containing the gene sequence(s), increasing the transcription of one or more endogenous genes that encode the one or more gene products, or by mutating a transcriptional control (e.g., promoter/enhancer) sequence of the one or more genes, or decreased, e.g., by introducing into the first cell nucleic acid molecules that reduce the expression of the one or more gene products such as nucleic acid molecules are, or express, siRNA molecules, or by mutating one or more genes that encode the one or more gene products or a transcriptional control (e.g., promoter/enhancer) sequence of the one or more genes.

Optionally, the changes in gene expression in the cell containing the mutated global transcription machinery are used to construct a model of a gene or protein network, which then is used to select which of the one or more gene products in the network to alter. Models of gene or protein networks can be produced via the methods of Ideker and colleagues (see, e.g., Kelley et al., Proc Natl Acad Sci USA 100(20), 11394-11399 (2003); Yeang et al. Genome Biology 6(7), Article R62 (2005); Ideker et al., Bioinformatics. 18 Suppl 1:S233-40 (2002)) or Liao and colleagues (see, e.g., Liao et al., Proc Natl Acad Sci USA 100(26), 15522-15527 (2003); Yang et al., BMC Genomics 6, 90 (2005)),

The invention also includes cells produced by any of the methods described herein. The cells are useful for a variety of purposes, including: industrial production of molecules (e.g., many of the tolerance phenotypes and increased metabolite production phenotypes); bioremediation (e.g., hazardous waste tolerance phenotypes); identification of genes active in cancer causation (e.g., apoptosis resistance phenotypes); identification of genes active in resistance of bacteria and other prokaryotes to antibiotics; identification of genes active in resistance of pests to pesticides; etc.

In another aspect, the invention provides methods for altering the production of a metabolite. The methods include mutating global transcription machinery to produce an altered cell, in accordance with the methods described elsewhere herein. The cell preferably is a cell that produces a selected metabolite, and as described above, preferably is previously optimized for production of the metabolite. Altered cells that produce increased or decreased amounts of the selected metabolite can then be isolated. The methods also can include culturing the isolated cells and recovering the metabolite from the cells or the cell culture. The steps of culturing cells and recovering metabolite can be carried out using methods well known in the art. Various preferred cell types, global transcription machinery and metabolites are provided elsewhere herein.

Another method provided in accordance with the invention is a method for bioremediation of a selected waste product. “Bioremediation”, as used herein, is the use of microbes, such as bacteria and other prokaryotes, to enhance the elimination of toxic compounds in the environment. One of the difficulties in bioremediation is obtaining a bacterial strain or other microbe that effectively remediates a site, based on the particular toxins present at that site. The methods for altering the phenotype of cells described herein represents and ideal way to provide such bacterial strains. As one example, bioremediation can be accomplished by mutating global transcription machinery of a cell to produce an altered cell in accordance with the invention and isolating altered cells that metabolize an increased amount of the selected waste product relative to unaltered cells. The isolated altered cells then can be cultured, and exposed to the selected waste product, thereby providing bioremediation of the selected waste product. As an alternative, a sample of the materials in the toxic waste site needing remediation could serve as the selection medium, thereby obtaining microbes specifically selected for the particular mixture of toxins present at the particular toxic waste site.

The invention also provides collections of nucleic acid molecules, which may be understood in the art as a “library” of nucleic acid molecules using the standard nomenclature of molecular biology. Such collections/libraries include a plurality of different nucleic acid molecule species, with each nucleic acid molecule species encoding a different mutated nucleic acid molecule. In some embodiments, such collections/libraries include a plurality of different nucleic acid molecule species, with each nucleic acid molecule species encoding global transcription machinery that has different mutation(s) as described elsewhere herein.

Other collections/libraries of the invention are collections/libraries of cells that include the collections/libraries of nucleic acid molecules described above. The collections/libraries include a plurality of cells, with each cell of the plurality of cells including one or more of the nucleic acid molecules. The cell types present in the collection are as described elsewhere herein. In the libraries of cells, the nucleic acid molecules can exist as extrachromosomal nucleic acids (e.g., on a plasmid), can be integrated into the genome of the cells, and can replace nucleic acids that encode the endogenous nucleic acids, such as the endogenous global transcription machinery.

The collections/libraries of nucleic acids or cells can be provided to a user for a number of uses. For example, a collection of cells can be screened for a phenotype desired by the user. Likewise, a collection of nucleic acid molecules can be introduced into a cell by the user to make altered cells, and then the altered cells can be screened for a particular phenotype(s) of interest.

Collections/libraries can be stored in containers that are commonly used in the art, such as tubes, microwell plates, etc.

In another aspect, the invention provides high throughput screens for isolating cells capable of high product accumulation, such as hyaluronic acid. In a preferred embodiment the high throughput screens utilize Alcian blue, a water soluble copper-phthalocyanine dye, C₅₆H₆₈C₁₄CuN₁₆S₄, which can be used for the staining of sulfated and carboxylated acid mucopolysaccharides (Penney et al., 2002). Hyaluronic acid is a mucopolysaccharide. The invention provides a two-step high throughput screen based on translucent colony identification in combination with alcian blue staining to quantify hyaluronic acid concentration.

From an evolutionary viewpoint, the potential of a strain improvement method is related to how effective it is for exploring the phenotypic space. This aspect can be measured using population diversity. Strictly, one should measure the diversity of a library, such as a sigma factor library at the transcriptomic level, but high-throughput analysis of the mRNA profile for thousands of samples is technologically unavailable. Alternatively, one may focus in diversity directly at the phenotypic level. This is an acceptable approximation as (i) it can be assumed that the phenotypic landscape as a function of the transcriptome is not perfectly flat, and (ii) we are more interested in feasible phenotypes than in feasible transcriptomes.

A quantification method has been also described for assessing the potential of different libraries for phenotype improvement. Any phenotype (e.g., growth rate under different conditions, metabolite production, internal pH, etc.) that can be assayed with a high-throughput screen can be used for quantification of phenotypic distance. For example, the intracellular pH (pHᵢ) is a complex trait that can be used, as it is affected by the relative levels of proteins and metabolites in the cell (Kresnowati M T A P, et al. 2007. Measurement of fast dynamic intracellular pH in Saccharomyces cerevisiae, using benzoic acid pulse. Biotechnology and Bioengineering 97: 86-98), and is expected to vary with changes in the transcriptome. In addition, pHᵢ is readily probed for individual cells using flow cytometry (Franck P, et al. 1996. Measurement of intracellular pH in cultured cells by flow cytometry with BCECF-AM. J Biotechnol 46: 187-95; Spilimbergo S, Bertucco A, Basso G, Bertoloni G. 2005. Determination of extracellular and intracellular pH of Bacillus subtilis suspension under CO2 treatment. Biotechnol Bioeng 92: 447-51).

The phenotype may be complex (such as those previously mentioned), but is not necessarily complex. For example, if one would want to quantify the variability of a promoter library that expresses green fluorescent protein, then the phenotypic value could be, for instance, fluorescence intensity. In other words, this method is useful generally to evaluate any library with a quantifiable phenotype, though high-throughput is preferred for practicability. The phenotype being measured is used to calculate the average phenotypic distance using,

d=<d_{i,j}>∀i,j

d_{i,j}=|Pᵢ−Pⱼ|

The value of d can be bootstrapped to find the distribution of its value. For normalization, statistical distance measures are used to subtract the distance value of a control population from that of the library population. The Bhattacharyya distance is an example of such a statistical distance measure.

This procedure can be used to compare the potential of libraries of different regulators (e.g., sigma S vs. Sigma D factors), different mutagenesis targets (−10 vs. −35 binding regions as described above), the effect on phenotype of different conditions, etc.

As an example of this approach, colony size under different conditions, related to growth rate, can be used as the complex phenotype used to quantify diversity. The average phenotypic distance between members of a population can be used to measure relative dissimilarity and to quantify the dimensions of the search space available to the population. When properly normalized, this distance reflects the divergence of a library (of a sigma factor or otherwise) with respect to the unmutated control. This method can be used to explore the effect of mutation frequency of a factor such as the sigma factor in phenotypic diversity, and to compare libraries such as sigma factor libraries to those prepared by NTG-mutagenesis.

The diversity quantification can be generalized to any random strain improvement method (genome-wide mutagenesis, transcriptional engineering, etc.) and to any directed evolution approach. In particular, it can aid at finding targets (e.g. proteins such as rpoD or rpoA or spt15, ribozymes, DNA-modifying enzymes, etc.) for strain improvement or even amino acids in those targets that have a high potential for improving phenotype. Thus the invention also provides methods for optimizing a cellular library such as wherein localized mutagenesis is applied to the library, and the level of phenotypic diversity is calculated, wherein the rate of mutagenesis is optimized to achieve maximum phenotypic diversity. This method can be iteratively performed for further optimization.

### EXAMPLES

### Materials and Methods

### DNA Manipulations, Plasmids, and Bacterial Strains

All DNA manipulations, such as genomic DNA isolation, restriction enzyme digestion and ligation, were performed by standard procedures (Sambrook et al., 1988) or following the specific manufacturer's instructions. Restriction enzymes were purchased from New England Biolabs, Taq DNA polymerase and primers were ordered from Invitrogen. Plasmid pMBAD (4093 bp) was constructed by the introduction of a 62 by multi-cloning sites (MCS) sequence containing XbaI-BamHI-StuI-KpnI-SacI-EcoRI-HindIII restriction sites into the plasmid of pBAD (Invitrogen) with an ampicillin resistance marker. E. coli Top10 (Invitrogen) was used as the expression host of the plasmid pMBAD-sseABC, which was constructed by the insertion of the fragment sseABC into the backbone of pMBAD. The sseABC is the abbreviation of the three genes sehasA, hasB and hasC. sehasA was synthesized by assembly PCR (Hoover and Lubkowski, 2002) according to the protein sequence of the HA synthase from Steptococcus equisimilis (NCBI-AAB87874.1, GI:2655100). hasB and hasC were the genes of ugd and galF in E. coli K12 MG1655, coding for the UDP-glucose 6-dehygrogenase and the glucose-1-P uridyltransferase, respectively. The ligation of ssehasA, hasB and hasC were carried out using the restriction sites of NcoI/XbaI, XbaI/StuI and StuI/KpnI, respectively. E. coli Top10/pMBAD-sseABC is an L-arabinose inducible recombinant E. coli strain for HA production, while E. coli Top10/pMBAD was used as the null control. E. coli DH5α (Invitrogen) was used for routine transformations as described in the protocol.

Library Construction

A low copy host plasmid (pHACM) was constructed as previously described (Alper and Stephanopoulos, 2007). The genes encoding the α subunit, the σ^{D} subunit and the σ^{S} subunit of RNA polymerase, denoted as rpoA, rpoD, and rpoS, respectively, were amplified from E. coli genomic DNA, using the following primers: rpoA-F-ApaLI: GCGCGCCCGGGACGTTGTAAGCATTCGTGAGAAAGCG (SEQ ID NO: 1) and rpoA-R-XmaI: GCGCGGTGCACTGGCGCATGACCTTATCCTCTCAGTA (SEQ ID NO: 2), rpoD-F-SacI: AACCTAGGAGCTCTGATTTAACGGCTTAAGTGCCGAAGAGC (SEQ ID NO: 3) and rpoD-R-HindIII: TGGAAGCTTTAACGCCTGATCCGGCCTACCGATTAAT (SEQ ID NO: 4), and rpoS-F-SacI: AACCTAGGAGCTCAGACTGGCCTTTCTGACAGATGCTTACT (SEQ ID NO: 5) and rpoS-R-HindIII: AACCTAGGAGCTCAGACTGGCCTTTCTGACAGATGCTTACT (SEQ ID NO: 6). Fragment mutagenesis was performed using the Genemorph® II Random Mutagenesis kit (Stratagene) with various concentrations of initial template to obtain low, medium, and high mutation rates as described in the product protocol as well as previously described (Alper and Stephanopoulos, 2007). Following the error-prone PCR, the mutated fragments of rpoA, rpoD and rpoS were purified using a Qiagen PCR cleanup kit, digested by the respective restriction enzymes overnight (ApaLI/XmaI for rpoA, HindIII/SacI for rpoD, HindIII/SacI for rpoS), ligated overnight into a digested pHACM backbone, and finally transformed into E. coli DH5α competent cells. Cells were plated on LB-agar plates and scraped off to create a liquid library. The total library size was approximately 10⁶. The plasmid library was extracted using the Qiagen Miniprep kit (Qiagen) and stored at −80° C. An approximately equal concentration of the plasmid library of pHACM-rpoA, pHACM-rpoD and pHACM-rpoS was transformed into E. coli Top10/pMBAD-sseABC by electroporation and plated on selective plates after dilution. The HA-producing libraries of Top10/(pMBAD-sseABC, pHACM-rpoA), Top10/(pMBAD-sseABC, pHACM-rpoD) and Top10/(pMBAD-sseABC, pHACM-rpoS) were abbreviated as HA-rpoA, HA-rpoD and HA-rpoS libraries, respectively.

Translucent Colony Screening Optimization

Different growth media were tested for optimizing colony formation phenotype of HA-producing strains. All media were prepared using the following concentrations of supplements, as specifically mentioned in each medium, MgSO₄.7H₂O, 0.25 g/L; MgCl₂, 0.95 g/L; sorbitol, 15 g/L; leucine, 0.2 g/L; L-arabinose (inducer), 0.1 g/L; amplicillin, 100 mg/L; chloramphenicol 34 mg/L. Six different modified media were used for optimizing the translucent colony screening, including M9^{M} (M9 supplemented with 10 g/L glucose, MgSO₄. 7H₂O, leucine, ampicillin, and L-arabinose), R^{M} (R medium (Wang and Lee, 1998) supplemented with leucine, ampicillin and L-arabinose), MOPS^{M} (MOPS medium (Teknova, Inc.) (Neidhardt et al., 1974) supplemented with leucine, ampicillin, and L-arabinose), MM1^{M} (MM1 medium (Bellemann et al., 1994) supplemented with MgSO₄.7H₂O, leucine, ampicillin, and L-arabinose), LBMA (LB medium supplemented with 15 g/L glucose, MgCl₂, ampicillin, and L-arabinose) and LBSMA (Bellemann et al., 1994) medium (LB Medium supplemented with sorbitol, MgCl₂, ampicillin and L-arabinose) were used for the medium optimization of the translucent colony screen.

### High Throughput Quantification of HA by Alcian Blue Staining

The alcian blue solution was prepared by the following procedure: 1.0 g alcian blue 8GX (Sigma Aldrich) was dissolved in 100 ml 3% glacial acetic acid and the pH was adjusted to 2.5 using acetic acid. The solution was filtered through a 0.45 μl syringe filter (VWR, USA), and a crystal of thymol was added; It was stored at room temperature and found to be stable for 6 months. The optimized procedure for high throughput HA quantification is as follows: 400 μl of fermentative broth containing HA was aliquoted into a 1.5 ml centrifuge tube pre-filled with 550 ul 3% acetic acid, 50 μl Alcian blue solution was added followed by vortexing, and the mixture was microwaved for 30 seconds; after centrifugation, the tube was cooled at room temperature for 2.5 h. Then, the solution was centrifuged at 10,000 rpm for 1 min, and 200 μl of supernatant were loaded into a 96-well plate, and the OD₅₄₀ was measured using the plate reader. A standard curve was generated using 400 μl of 50, 100, 200, 300 and 500 mg/L commercial HA standards (VWR, USA). All experiments were repeated 3 times except where specially noted.

### Specific HA Titer Measurement by HPLC Method

HA titers were measured by the modified HPLC method (Kakizaki et al., 2002). Fermentation broth samples were incubated first with an equal volume of 0.1% w/v sodium-dodecyl-sulfate (SDS) at room temperature for 10 min to free the capsular HA (Chong and Nielsen, 2003). Subsequently, the HA product was precipitated out from the medium samples with 1.5 volumes of ethanol (Ogrodowski et al., 2005) incubating at 4° C. for 1 h. The precipitate was collected by centrifugation (2,000 g for 20 min at room temperature) and resuspended in 1 volume of 0.2 M NaCl for 10 min. Then the re-dissolved samples were centrifuged for 8 min at 3000 g, filtered through a 0.45 μl syringe filter (VWR, USA), and applied to the modified HPLC assay. Gel Filtration Chromatography (GFC) in combination with a UV photodiode array detector (Waters 2695-996) was used to determine the concentration of the HA products in the broth. The column was a model Shodex SB-806M OHpak (8×300 mm, Thompson, USA) supporting Mw analyses from 10³ to 2×10⁷ Da. HA products at Mw of 6.8×10⁵ Dalton, purchased from Lifecore Biomedical Inc., were prepared into around 300 mg/L aqueous standards in 0.2 M NaCl. The detection was carried out at wavelength of 206 nm and room temperature, with 0.2 M NaCl as the effluent buffer at flow rate of 0.5 ml/min.

### Phenotype Selection, Media and Culture Conditions

LBSMA^{C} solid medium was used for the translucent colony screening of HA-producing libraries using the LBSMA medium further supplemented with chloramphenicol. Selected translucent colonies were transferred to 2 ml LB^{AC} medium cultures and cultured overnight in 30×115 mm closed top centrifuge tubes shaking at 37° C. 2% (V/V) inoculums of the stationary phase culture were used to culture the selected clone in another tube with 1 or 2 ml LBM^{AC} medium (LB medium supplemented with MgCl₂, ampicillin and chloramphenicol). These cultures were incubated at 37° C. for 2.5 h (OD₆₀₀˜0.8), induced with L-arabinose. After 5 hrs, the cultures were supplemented with 10 g/L glucose to allow accumulation of HA. Cultures were stopped at 24 h, and HA concentration was quantitatively measured by the alcian blue method in a 96 well plate measuring OD₅₄₀ by a Packard Fusion 96 well plate reader. For one batch of screening, usually 38 transparent library colonies were simultaneously quantified with 2 dense colonies of original Top10/pMBAD-sseABC as a control.

The optimal selected HA-rpoA, HA-rpoD and HA-rpoS library strains were plated, inoculated, and cultured in 40 ml LBM^{AC}/250 ml flasks at 37° C. with 225 RPM orbital shaking for further HA productivity testing. Cell density was monitored spectrophotometrically at 600 nm by an Amersham Biosciences Ultraspec 2100 Pro. The inducer of 0.1 g/L L-arabinose was added at around 2.5 h when OD₆₀₀ reached 0.8. Glucose (10 g/L) was later supplemented at 5 h. Further glucose (6 g/L) was supplemented at 30 h, and the pH of the broth was adjusted to 7.0-7.5 using NaOH (4 mol/L) at 24 h and 30 h, respectively. Broth was harvested at 48 h to assay the specific HA titer using the HPLC method.

### Example 1

### A) Translucent Colony Formation and Identification of HA-Producing Recombinant E. Coli

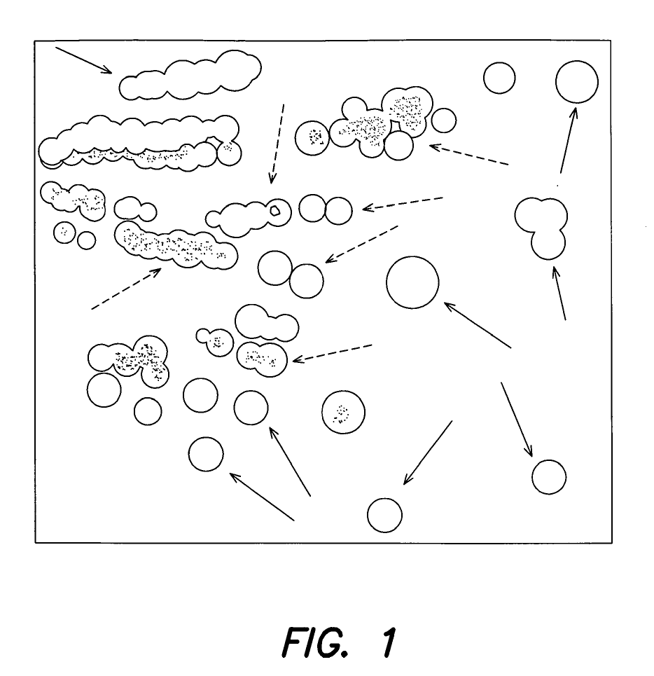

In light of the conventional method typically used to identify high-HA-producing strains of Streptococcus spp., and B. subtilis, by viscous colony morphology on solid medium (Kim et al., 1996; Widner et al., 2005), screening based on colony-morphology was also employed for identifying HA-producing cells in recombinant E. coli. As listed in Materials and Methods, six modified media denoted as M9^{M}, R^{M}, MOPS^{M}, MM1^{M}, LBMA and LBSMA, were tested for mucoid or other special colony morphology due to the secretion of HA. Results showed that both the HA-producing strain, Top10/pMBAD-sseABC, and the non-HA producing strain, Top10/pMBAD, could not grow well on M9^{M}, R^{M} and MOPS^{M} solid media. Colonies of Top10/pMBAD-sseABC appeared on MM1^{M} plates after 3 days incubation at 37° C., but did not show any special morphological traits compared to Top10/pMBAD. Similar results were observed for LBMA solid medium. However, for the sorbitol-containing medium of LBSMA, the translucent colonies of Top10/pMBAD-sseABC were apparently different from the dense colony morphology of Top10/pMBAD. As shown in FIG. 1, overnight cultures of Top10/pMBAD-sseABC and Top10/pMBAD plated simultaneously on LBSMA formed notably different colonies with translucent or dense morphology, respectively. The observed difference between the two types of strains can be used for qualitative identification of HA-producers in recombinant E. coli. Further studies showed that translucent morphology can be observed with strains producing as little HA as 50 mg/L. It appears that the higher the HA productivity of the cells, the more transparent the colonies they form.

### B) High Throughput Quantification of HA-Producing Strains using Alcian Blue Staining

While colony morphology is adequate to discern large differences in HA productivity, a more quantitative approach is necessary to screen for incremental improvements in HA accumulation. Therefore, a novel screening method designated alcian blue staining was developed, that is scalable in throughput and significantly more quantitative in predicting HA titer.

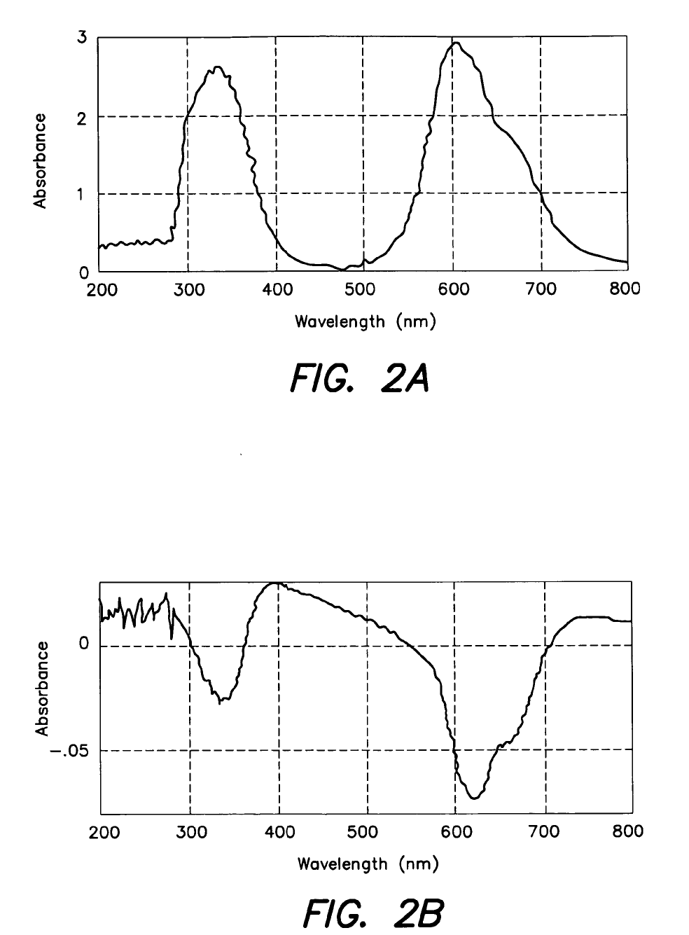

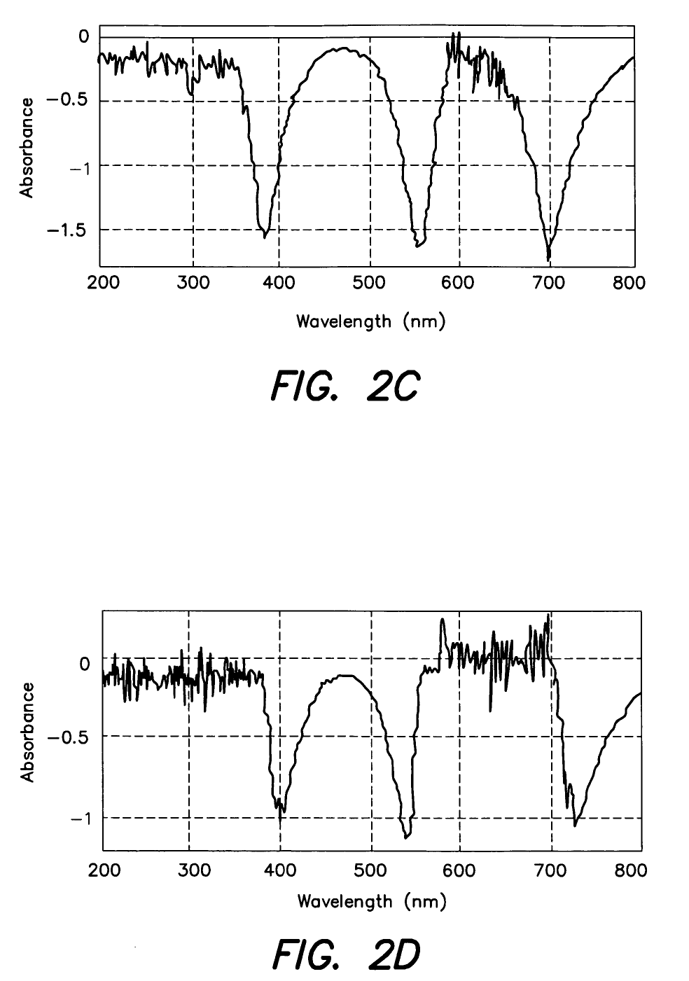

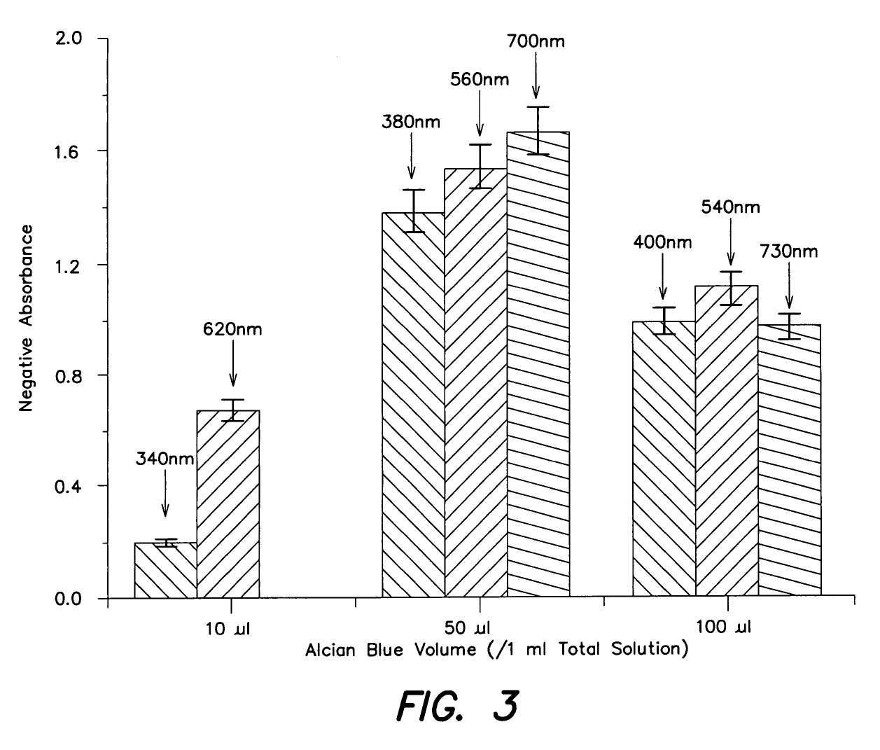

Absorbance scan of a pure alcian blue solution from 200 nm to 800 nm yields two positive absorbance peaks at 334 nm and 605 nm (FIG. 2a). However, after adding 10 μl, 50 μl and 100 μl alcian blue solution into 1.0 ml of 200 mg/L HA and using the corresponding alcian blue solution without HA as blank control, the absorbance pattern was significantly changed showing decreased absorbance at different wavelengths (correspondingly FIGS. 2b, 2c and 2d), such as 380, 560 and 700 nm in the 50 μl alcian blue mix. Furthermore, a visible precipitation of alcian blue was observed. By comparing the absorbance intensities of each peak it was found that the 50 μl alcian blue staining system showed the strongest negative absorbance relative to the solution without HA (FIG. 3), and it was thus selected as the optimal concentration for subsequent experiments.

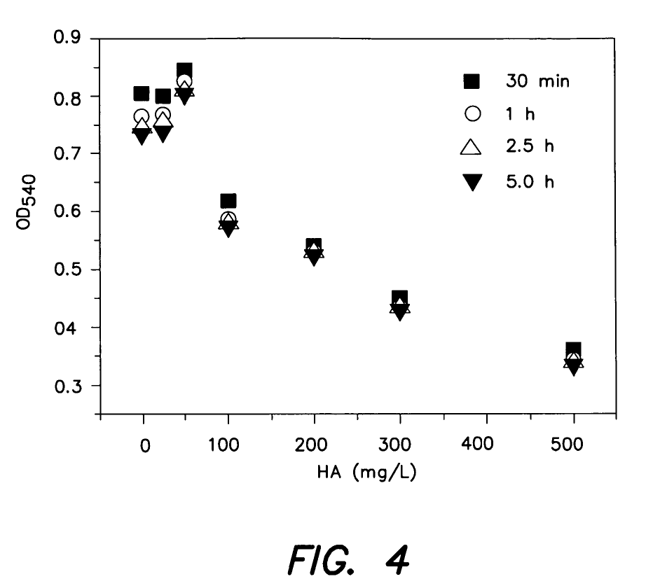

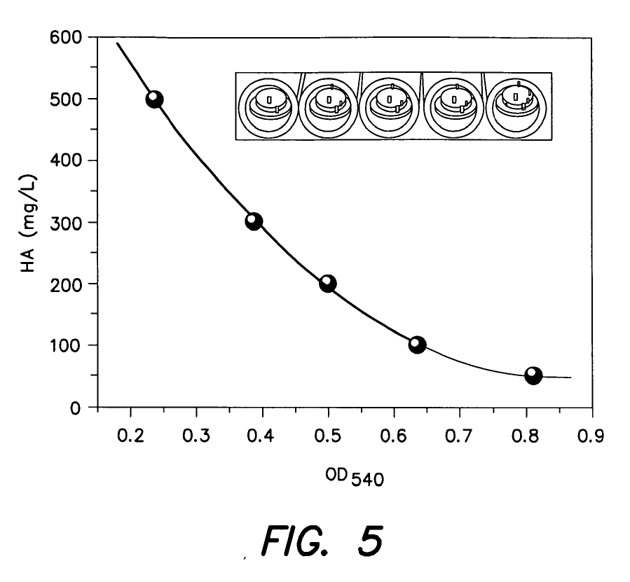

Subsequently, absorbance using the 540 nm filter for 50 μl/ml alcian blue staining was measured at six different HA concentrations to test for linearity at the following HA standard: 25, 50, 100, 200, 300 and 500 mg/L HA. As can be seen in FIG. 4, a second-order polynomial formula fits the HA-OD₅₄₀ response curve in the range of 50-500 mg/L HA concentration, and a linear fit can be observed from 100-500 mg/L HA in alcian blue if incubation is increased beyond 1 h. By setting the HA-alcian blue binding time at 2.5 h and fitting the OD₅₄₀-HA using a second-order polynomial formula (FIG. 5), a correlation of R²˜0.99945 was obtained indicating that the alcian blue staining method can quantitatively predict HA concentrations.

### Example 2

### A) High Throughput Screening of HA-rpoA, HA-rpoD and HA-rpoS Libraries

The above screen was applied to the identification of sigma factor mutants eliciting increased HA production in the previously engineered E. coli. Here, we also mutated the α subunit of the core RNA polymerase (RNAP) which has been shown to contribute to DNA recognition through interactions in sequences upstream of the canonical −35 promoter region (Ishihama, 1992; Busby and Ebright, 1994). The α subunit may interact directly with DNA or with activators or repressors of transcription, and thus helps modulating the relative mRNA abundance in the cell (Chen et al., 2003). Additionally, libraries of the σ^{D} factor (Alper and Stephanopoulos, 2007) which controls the expression of around 1,000 genes responsible for normal exponential growth (Gregory et al., 2005; Heimann and Chamberlin, 1988), and the σ^{S} factor that orchestrates the stationary phase phenotype in response to cessation of growth caused by various stresses (Venturi, 2003) were also screened. By random mutagenesis of the genes rpoA (coding for α), rpoD (coding for σ^{D}) and rpoS (coding for σ^{S}), three libraries were constructed, transformed into the parental HA-producing strain and screened for HA production as described in the Materials and Methods section. Each library has three levels of mutation frequency, denoted as high (H), moderate (M) and low (S).

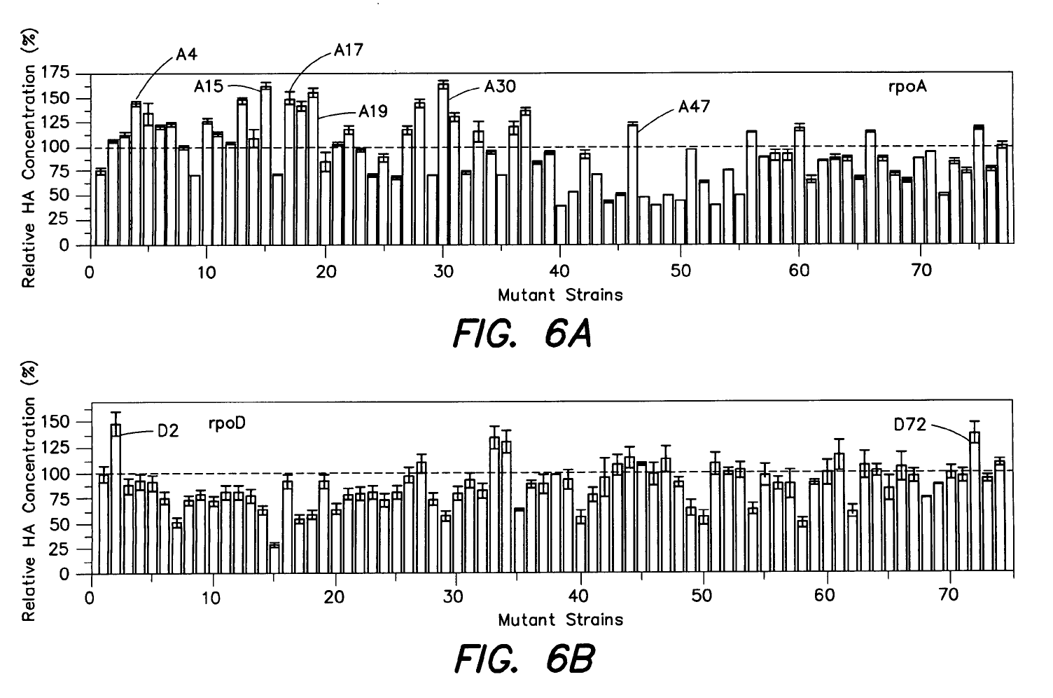

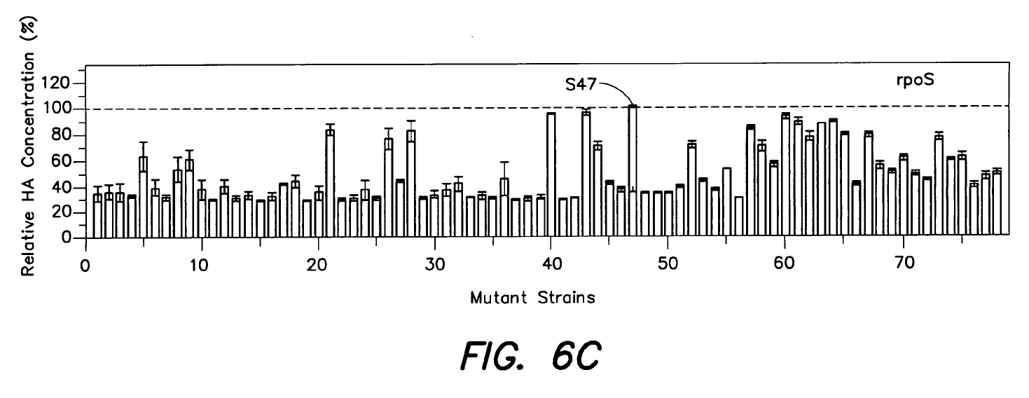

Using the first identification step (translucency), 77 rpoA mutants, 74 rpoD mutants and 78 rpoS mutants were selected from thousands of colonies on solid plates, and subsequently tested for HA accumulation by the alcian blue method. The parental strain carrying only the plasmid for HA synthesis (Top10/pMBAD-sseABC) was simultaneously cultured and used as a control. The selection results are plotted in FIG. 6, in which the A4, A5, A15, A17, A30 and A47 strains in the HA-rpoA library, and the D2 and D72 strains in the HA-rpoD library showed a significant increase of HA concentration relative to the control (100% line). Most mutants in the HA-rpoS library caused a decrease in HA accumulation while only one strain, S47, was slightly improved. This result is reasonable considering that σ^{S} is a stationary phase transcription factor, and might not be helpful for cell growth and HA accumulation within 24 h of inoculation. However, the HA-rpoA and HA-rpoD libraries were effective in eliciting E. coli phenotypes with improved HA production.

### B) Further Characterization of Improved Recombinant E. coli Mutants for High-HA Production

The most promising mutants obtained from the primary screening described above were further studied in shake flask cultures. The culture volume was scaled up to 40 ml medium in a 250 ml flask. Strains A4, A15, A17, A19, A30, A47, D2, D72 and S47 were simultaneously cultured for 48 h, and the HA titer per cell weight was measured to evaluate the HA-producing capability of the mutants, as shown in Table 1.

TABLE 1
Comparison on cell growth and HA accumulation characteristic
of the selected mutants from the libraries.

| | | | Specific HA | | |
|---|---|---|---|---|---|
| | DCW | HA titer | productivity | Productivity | Selection |
| Strains | (g/L) | (mg/L) | (mg HA/g cell) | Increase (%) | evaluation |
| Control | 2.26 | 197.7 ± 8.8 | 87.5 ± 3.6 | / | / |
| A4 | 1.86 | 166.5 ± 7.4 | 89.4 ± 4.0 | 2.1 | / |
| A15 | 1.88 | 179.0 ± 8.0 | 95.4 ± 4.2 | 9.0 | ++ |
| A17 | 1.83 | 169.8 ± 7.6 | 92.8 ± 4.1 | 6.1 | + |
| A19 | 1.84 | 175.3 ± 7.8 | 95.3 ± 4.2 | 8.9 | ++ |
| A30 | 1.61 | 164.9 ± 7.3 | 102.2 ± 4.5 | 16.8 | +++ |
| A47 | 1.82 | 172.6 ± 7.7 | 94.9 ± 4.2 | 8.5 | ++ |
| D2 | 2.08 | 171.0 ± 7.6 | 82.3 ± 3.7 | −6.0 | / |
| D72 | 1.72 | 174.8 ± 7.8 | 101.5 ± 4.5 | 16.0 | +++ |
| S47 | 1.88 | 183.9 ± 8.2 | 98.0 ± 4.4 | 12.0 | +++ |

Note:
Two parallel experiments were carried out and the culture conditions were described in Materials and Methods. The control strain is the Top10/pMBAD-sseABC, and the HA titer was measured by HPLC. The retention time of the peaks were around 15.6 min, which corresponds to HA's MW of 5.0-6.0 × 10⁵ Dalton.

It can be seen that all mutants reached lower biomass levels presumably due to the increased cell burden from the higher HA synthesis and double plasmid replication. Less biomass correspondingly yielded lower final HA titer. Relatively, strain A30 and D72 showed the highest specific productivity of HA (˜16% increase in comparison with control), an interesting result for extending the uses of these libraries. Strain S47 exhibited the highest HA titer although it showed the lowest improvement during the library screening. This was probably due to the extension of the culture time from 24 to 48 h, allowing cells to reach stationary phase and fully express the mutated σ^{S} factor. It is reasonable to expect that under optimized fed-batch culture conditions, these three strains, D72, A30 and S47, can achieve high HA accumulation simultaneously with high cell density, therefore deserving further studies on their fed-batch fermentation.

### Example 3

### RpoA Mutant Strains with Enhanced Capacities for L-Tyrosine Production

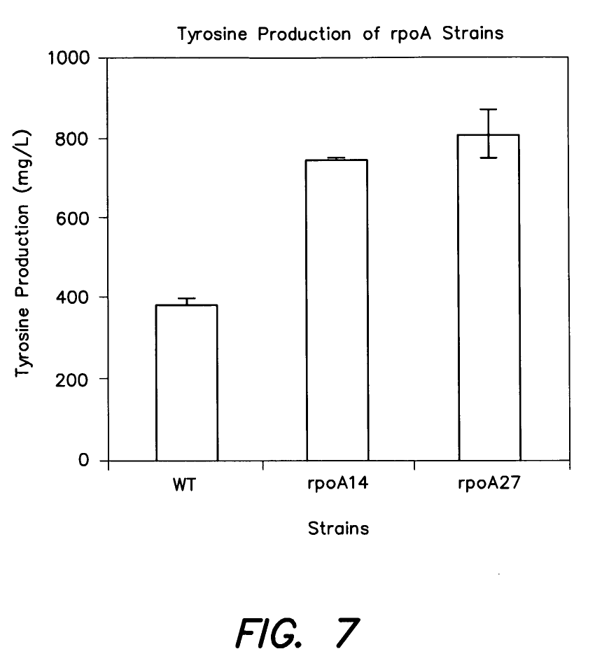

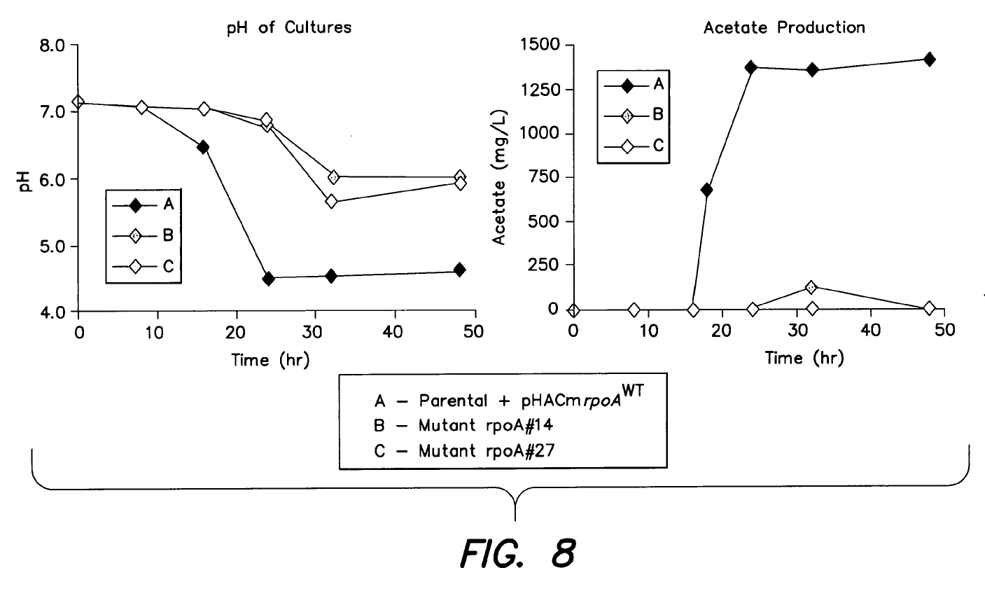

pHACm-rpoA plasmid libraries were transformed into E. coli K12 ΔpheA tyrR::P_{LtetO-1}tyrA^{fbr}aroG^{fbr}lacZ::P_{LtetO-1}tyrA^{fbr}aroG^{fbr}, a parental strain containing chromosomal overexpressions of two key genes in the aromatic amino acid biosynthetic pathway. Libraries on the order of 10⁶ in size were screened with a melanin-based assay outlined in “Methods for Identifying Bacterial Strains that Produce L-tyrosine” (U.S. Provisional Application No. 60/965,149). From this search, two mutant strains—rpoA14 and rpoA27—were isolated which exhibited tyrosine production levels up to 96 and 112% above the parental strain, respectively (FIG. 7). FIG. 8 shows the concurrent change of pH (A) and the change of acetate production (B) in medium over time when the rpoA mutants strains rpoA14 and rpoA27 or the rpoA-wt parental strains were cultured for up to 48 hours.

### Example 4

### RpoA Mutant with Higher Solvent Tolerance

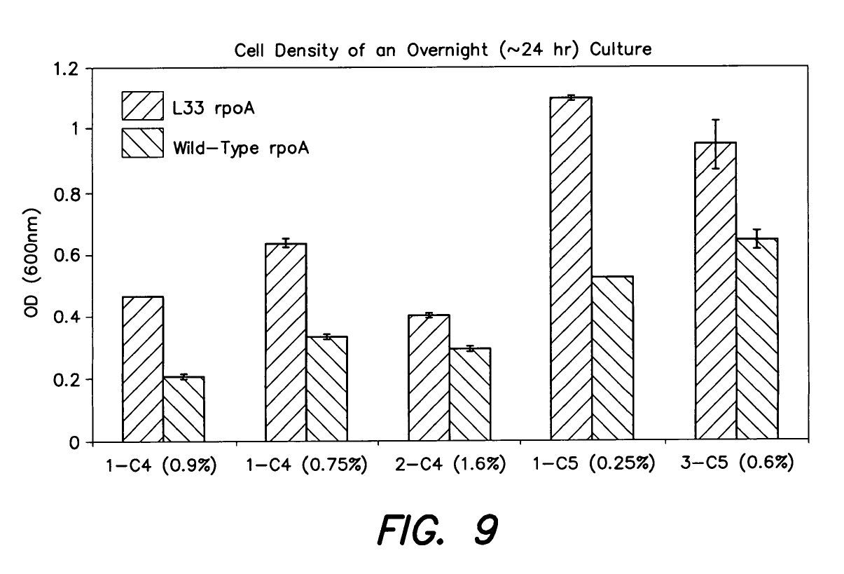

The same rpoA library described above was transformed into E. coli and screened for growth in butanol. An improved mutant (L33) was obtained that grew better than control in several solvents as shown in FIG. 9. The graph shows overnight growth of DH5α cells transformed with either the wild-type or the L33 mutant of rpoA in different alcohol solvents. Solvent tolerance is a significant phenotype, as many biofuels have solvent properties, and their mass production may be limited by toxicity.

### Example 5

### Phenotypic Diveristy for Optimizing Random Strain Improvement Libraries

Random searches have been the hallmark of directed evolution. In the context of cellular engineering, they have been extensively employed in the improvement of complex or poorly-understood phenotypes, such as metabolite overproduction or tolerance to toxic compounds (Santos and Stephanopoulos, 2008). A sustainable economy will depend on efficient renewable-feedstock conversion to chemicals and fuels, and advances in that direction have relied and will continue to rely on cellular engineering (Lynd et al. 1999). In this regard, genome-wide mutagenesis followed by screening has been a traditional means of improving phenotype (Demain, et al. 1999; Rowlands 1984), but the list of experimental methods for cellular engineering based on random searches is rapidly expanding (Alper, et al. 2006; Beltran, et al. 2006; Jin and Stephanopoulos 2007; Miyagishi et al. 2005; Park et al. 2003). Adding to the confusion is the element of chance, which limits the information that can be derived from both successful and failed attempts to isolate improved strains. We hereby present a method for obtaining such information based on quantification of phenotypic diversity, and describe its use for optimizing cellular libraries to arrive at improved strains.

Random searches for phenotypic improvement, similar to the iterations of directed evolution, comprise two steps: introducing genetic diversity and screening for variants with interesting traits. Because most protocols for introducing genetic diversity hinge on creating combinatorial arrangements of many nucleotides, the number of variants that can be constructed is virtually infinite. This implies that in most cases we cannot cover the search space experimentally, which becomes a particularly relevant problem when screening for phenotypes of interest fails to deliver improved variants. In this case, the result of one experiment rarely suggests ensuing experiments, because it is difficult to ascribe the failure to particular steps of the random search protocol. This changes if we can evaluate and improve the libraries themselves; a good library in this sense is one in which there is a high probability that a useful phenotype can be found. A central difficulty raised by this definition is that it is not a priori specified what traits are of interest, because the libraries can be screened for improvement of different and even distant phenotypes (Alper and Stephanopoulos 2007; Klein-Marcuschamer et al. 2008; Park et al. 2003). Therefore, to have a higher a priori probability of harboring a mutant with an improved trait, a library must be phenotypically diverse (Klein-Marcuschamer and Stephanopoulos 2008).

Increasing the probability of success during screening presents notable economic advantages, as it is known to be a key time- and labor-intensive step (Demain et al. 1999; Kittell et al. 2005). This is especially true for increasing the production of metabolites, where screening generally involves fermentation of thousands of individual mutants followed by mass spectroscopy, liquid chromatography, or similar analytical techniques (Stutzman-Engwall et al. 2005). Adding to the cost is the fact that substantial time and expense can be incurred before the researcher realizes that the ongoing method has little chance of delivering improved mutants (Demain et al. 1999). Selection for tolerance to toxic products or to anti-metabolites is less expensive, but it is a poorly-understood process and many parameters can be manipulated (choice of medium, concentration time-profile of the toxic compound, parameters such as pH and temperature, etc.) (Bonomo et al. 2008; Warnecke et al. 2008). If many libraries are to be screened, this lengthy and uncertain process translates into significant expenses.

We hypothesized that optimization of strain improvement libraries aimed at probing the search space by targeting mutagenesis could increase the probability of finding a desired mutant or could aid directing the construction of better libraries. A nontrivial tradeoff of reducing the search space is that potentially useful mutations are forgone by restricting the nucleotide regions that are allowed to be changed. An ideal route towards optimization of a library would be delimiting the search space by ignoring genetic determinants that when altered result in phenotypically redundant variants, but keeping those that result in new phenotypes. As with any optimization algorithm, a metric is needed to evaluate whether progress is made at each step of the process.

We recently reported a method for quantifying the evolutionary potential of random libraries that could serve this purpose (Klein-Marcuschamer and Stephanopoulos 2008). The diversity metric (called divergence) thereby developed conveys how much more different, on average, are members of a library population to each other, compared to how different are members of a clonal, wild-type population to each other with respect to a complex trait (i.e. one that results from the interplay of many intracellular components). In some sense, we use variability in a complex phenotype, such as growth rate or intracellular pH (pHᵢ), as a proxy for how “reachable” are novel phenotypes in general, and we have shown that this variability correlates with the probability of finding an improved strain (Klein-Marcuschamer and Stephanopoulos 2008). Implicit is the assumption that the mutagenesis protocol alters the physiological network globally (e.g. by targeting a central node of the network (Martinez-Antonio et al. 2008)), and thus diversity in a measurable complex phenotype is tied to diversity in other (immeasurable) phenotypes. The pHᵢ can be used for quantification of divergence, as it is affected by the relative levels of proteins and metabolites in the cell even when it is maintained in a narrow range (Kresnowati et al. 2008).

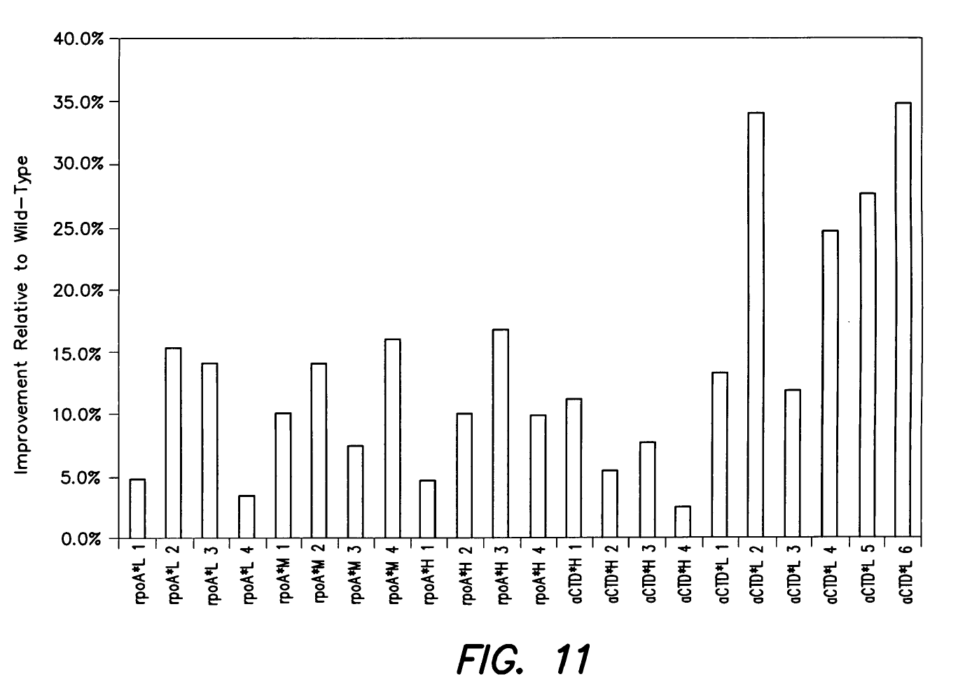

We have been working with a random strain improvement method that is based on global alteration of the transcriptome and has delivered several improved mutants (Klein-Marcuschamer et al. 2008; Yu et al. 2008). In the present study, we used the alpha subunit of the RNA polymerase (RNAP) as our target for cellular engineering. Mutations in this protein can perturb transcription profiles globally as it is thought to act at most, if not all promoters (Ross and Gourse 2005). Previously, we built three libraries that varied in their mutation frequency (denoted rpoA*L, rpoA*M, and rpoA*H) and successfully used them to isolate strains with improved butanol tolerance, hyaluronic acid accumulation, and tyrosine production (Klein-Marcuschamer et al. 2008). We were also interested in a butyrate-tolerant mutant, because this compound can be used to produce butanol (in a two-step fermentation (Tashiro et al. 2004) or catalytic reduction) and propane (Fischer and Peterson, WO2008/103480), both of interest as renewable fuels. The toxicity of butyrate is thought to arise from dissipation of the pH transmembrane gradient, similar to other weak acids, although limited research has been conducted in this regard (Zigova and Sturdik 2000). When we screened the same libraries that had resulted in several improved phenotypes in the presence of butyrate, we failed to isolate tolerant strains, even after many experimental conditions were tried (FIG. 11). We deemed this a perfect test case for applying the divergence metric to guide the design and construction of better libraries.

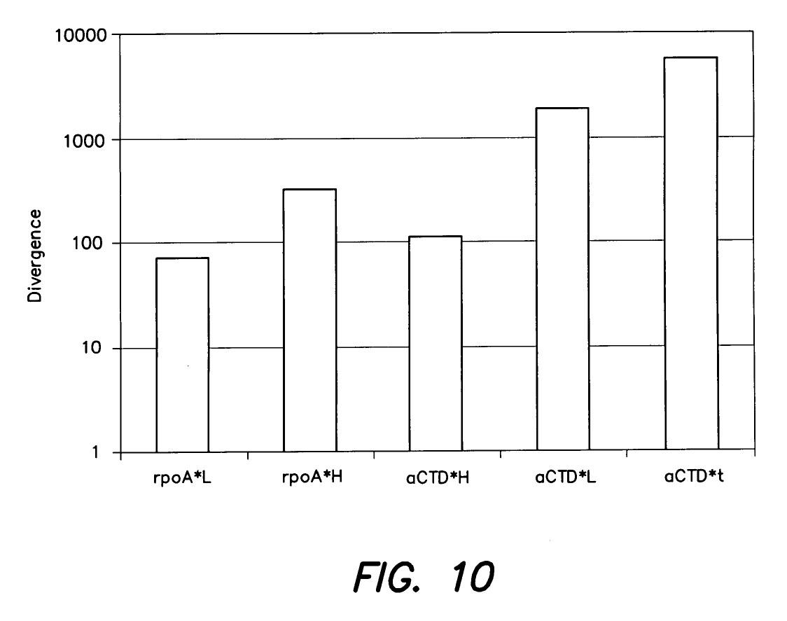

As a first step towards implementing our library optimization method, we quantified the diversity in the rpoA*L and rpoA*H libraries, which we had extensively screened in butyrate, albeit with no results. As shown in FIG. 10, there is an increase in divergence when sequence diversity in rpoA is increased, but our inability to find improved mutants suggested that a new, more phenotypically diverse library was needed. Our previous study on the alpha subunit resulted in three improved mutants, all of which had nucleotide changes in the αCTD (Klein-Marcuschamer et al. 2008). Therefore, we hypothesized that diversity could be increased by directing mutagenesis to this region of the protein. We constructed a library in which this region was mutagenized with high frequency, after observing that highest phenotypic diversity is accomplished with extensive mutagenesis (FIG. 10, Klein-Marcuschamer and Stephanopoulos 2008, and unpublished observations).

Quantifying the phenotypic diversity of the new library (denoted αCTD*H) contradicted our expectations (FIG. 10). Not only did the diversity not increase by focusing the mutations to the αCTD, but it actually decreased. Although the prospect of finding an improved mutant in this library was low, we screened in butyrate to test our strategy. This screening step could have been eliminated if time was of essence or if the protocol was too costly (see supplementary information). Four independent selection experiments confirmed our expectations; we were unable to isolate improved mutants, thus a new library was needed.

We thought of two possible explanations for the decrease in diversity in αCTD*H compared to rpoA*H: (i) that by focusing the mutations to this domain we lost diversity because mutations in the N-terminal domain (αNTD) also confer novel phenotypes (e.g. by modulating the assembly of RNAP complexes or by transcriptional regulation at class II promoters (Niu et al. 1996)); or (ii) that the mutation frequency was too high, and that the diversity was lost because when a useful mutation was obtained, its effect vanished due to subsequent mutations. In other words, high mutation frequencies may reduce the diversity in our library because many clones display the same phenotype: that of expressing an alpha subunit with a non-functional CTD. To test these hypotheses, we constructed a library in which the mutagenesis is focused to the αCTD, but with lower mutagenesis rate (denoted αCTD*L). Quantifying the diversity of this library favored the second hypothesis (FIG. 10). This library has in fact higher diversity than that of the rpoA library with high mutation frequency throughout the coding region (rpoA*H). The mutation frequency in the CTD of rpoA*H is comparable to that of αCTD*L, but the latter has markedly more diversity. Thus, the most likely explanation for the diversity in rpoA*H is that it arises from changes in the αCTD in the context of an αNTD that is not entirely robust to mutations. Previous studies have described several mutations in αNTD that preclude its association into functional RNAP complexes (Kimura and Ishihama 1995), which seems a likely cause for the difference in diversity between rpoA*H and αCTD*L.

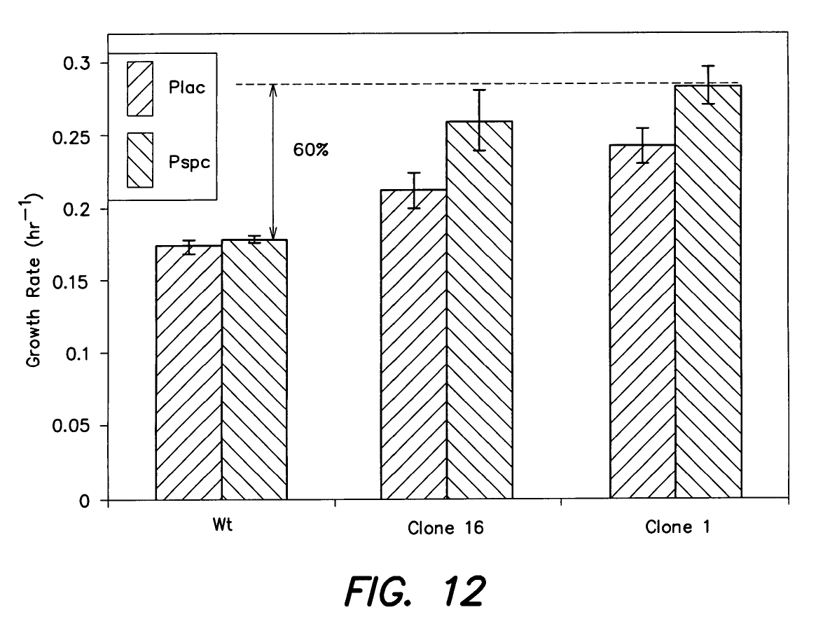

When we screened the αCTD*L library in the same conditions that were tried with our previous libraries, two improved mutants were finally isolated (FIG. 11). The mutants show a 23% and 40% improvement in growth rate in the presence of 15 g/L butyrate (FIG. 12). Not coincidentally, the two mutants have the same amino acid sequence and only one amino acid change with respect to the wild-type (S299T), consistent with the diversity assessment that small changes in sequence in the αCTD result in large changes in phenotype. Amino acid S299 is directly involved in interacting with UP promoter elements (Gaal et al. 1996); therefore, the mutation should alter the affinity of the RNAP for several targets, resulting in the novel phenotype. The mutant with lower improvement (23%) differs from the mutant with higher improvement (40%) in a synonymous substitution that changes a codon that is frequently used in E. coli (GGT for glycine) with an unusual codon (GGA). With this in mind, we placed the mutant and wild-type genes under a stronger promoter (P_{spc}) to see whether we could increase the growth rate further, and we obtained an up to 60% improvement. This advantage is substantial, considering that productivity of a metabolite in a continuous reactor is related to growth rate. We also analyzed the posterior probability of finding the S299T mutant in the different screened libraries and found that it was highest for αCTD*L (see supplementary material). This showed that obtaining the improved clone from this library was not accidental, but an even more compelling case for the information contained in the divergence metric is the fact that all mutants that have been isolated up to date have 1 or 2 mutations in the αCTD (Klein-Marcuschamer et al. 2008).

Although the goal of isolating an improved mutant had been achieved, we had gathered enough information to optimize our libraries further. Given that the diversity of αCTD*L is higher than that of αCTD*H, we hypothesized that this domain of the protein is very sensitive to mutations. Non-specific amino acid changes may prevent the αCTD from folding properly so that it cannot attain the conformation necessary for interacting with promoters. This suggested the construction of a library in which mutations were restricted to surface amino acids of this domain, thereby introducing diversity and at the same time preventing the formation of many non-functional, unfolded variants. As shown in FIG. 10, one such library (αCTD*t) lead to a marked increase diversity. The choice of amino acids was suggested by structural information (Jeon et al. 1995) and previous studies (Murakami et al. 1996) (see Materials and Methods), but our selection is most probably sub-optimal. Future efforts will aim at selecting and evaluating different combinations of amino acids in search of a better set.

Using the divergence metric that was previously developed, we have shown that random searches for strain engineering can be (semi-) rationally directed. In essence, the method here presented relies on successively evaluating the search space prior to screening for a particular phenotype. The methodology can be used not only to accelerate and economize strain improvement programs by eliminating screening steps with low probability of success, but also to direct the construction of libraries (as was the case for αCTD*L and αCTD*t). That is, one can probe the characteristics of the search space and potentially use this information for designing better populations. In addition, comparing the diversity of several libraries can be used to propose mechanistic explanations for such differences. Ultimately, the goal would be to gather enough information about a particular target and sequentially reduce the search space to the point where it can be widely covered.

### Materials and Methods

### Strains and Library Construction

Escherichia coli K12 recA⁻ as used throughout the study, except for transformation of the ligation reactions. The native rpoA gene was amplified from genomic DNA using Phusion DNA polymerase (Finnzymes) with primers A and B and cloned into the ApaLI and XmaI sites of the multi-cloning site of pHACm (Alper and Stephanopoulos 2007), using NEB restriction enzymes as in Klein-Marcuschamer et al. 2008. The correct insert was verified by sequencing and strains transformed with this plasmid are denoted ‘wild-type’ throughout the study. For rpoA*L, rpoA*M, and rpoA*H, error-prone PCR was carried out with the same primers using the GeneMorph II kit (Stratagene), resulting in approximately 4, 7, and 9 mutations/kb, respectively. For αCTD*H and αCTD*L, a BsiWI restriction site was introduced by a point mutation T707C (slightly upstream of the CTD) using a QuikChange Multi Site-Directed Mutagenesis Kit (Stratagene). The CTD sequence was amplified by error-prone PCR with primers B and C (resulting in ˜5-6 and ˜1-2 mutations per sequence, respectively) and cloned between the newly-introduced BsiWI and the ApaLI present at the 3′-end. For the αCTD*t library, two oligonucleotides (D and E) spiked at the target positions with 6% non-wild-type bases were constructed, and an artificial BglII site was introduced at the 5′-end of each primer to allow for re-circularization of the plasmid (the BglII site was introduced by a T835A mutation between amino acids E273 and E286). The residues targeted for mutagenesis in αCTD*t were: D259, L262, R265, N268, C269, K271, E273, E286, L290, G296, K298, and 5299. The entire plasmid was amplified with Phusion DNA polymerase using the spiked oligonucleotides D and E and cut with BglII and DpnI to rid the mix of the unmutated plasmid. Neither BsiWI nor BglII sites changed the amino acid sequence of rpoA. The primers are the following (restriction sites are underlined and a star implies the preceding base is spiked):

(SEQ ID NO: 7)

| | |
|---|---|
| A: | 5′-GCGCGCCCGGGACGTTGTAAGCATTCGTGAGAAAGCG-3′ |

(SEQ ID NO: 8)

| | |
|---|---|
| B: | 5′-GCGCGGTGCACTGGCGCATGACCTTATCCTTCTCAGTA-3′ |

(SEQ ID NO: 9)

| | |
|---|---|
| C: | 5′-ACGTGACGTACGTCAGCCTGAAGTGAAAGAAGAGAAACC-3′ |

(SEQ ID NO: 10)

| | |
|---|---|
| D: | 5′-TATCGGAGATCTGGTACAGCGTACCG*A*G*GTTGAGCTCC* |
| | T*T*AAAACGCCTAACCTTG*G*T*AAAA*A*A*T*C*T*CTTAC |
| | TGAGATTAAAGACGTGCTGGCTTCCCGT-3′ |

(SEQ ID NO: 11)

| | |
|---|---|
| E: | 5′-TGTACCAGATCTCCGATATAGTGGATACGT*T*C*TGCT*T* |
| | T*AAGG*C*A*G*T*T*AGCAGAG*C*G*GACAGTC*A*A*TTCC |
| | AGA*T*C*GTCAACAGGGCGCAGCAGGATCGGAT-3′ |

All ligations were done using Fast-link ligase (Epicentre) and transformed into DH10B cells (Invitrogen), which were plated in LB agar and pooled together after overnight growth. The plasmids were recovered by miniprep (Qiagen) and used to re-transform the K12 recA⁻ host strain. Each library was approximately 10⁵ in size. K12 recA⁻ cells were grown in MOPS (Teknova) or M9 (US Biologicals) minimal media with 0.5% glucose (unless noted) and the plasmid-borne rpoA was induced with 1 mM IPTG when measuring pHᵢ or during selection in butyrate. Chloramphenicol (34 μg/mL) and streptomycin (50 μg/mL) were added as needed.

### Diversity Quantification Using Intracellular pH

The divergence metric is calculated by measuring the pair-wise phenotypic distance between members of a library population, averaging it, and normalizing it with that of the control population. The divergence for each library can be calculated from the distance in several phenotypes, each constitutes an entry in the phenotypic distance vector used for calculating divergence (Klein-Marcuschamer and Stephanopoulos 2008). This ensures that the result is not biased by a particular dataset. In this study, we used the intracellular pH in growing and non-growing cells as phenotypes contained in the divergence metric. For determination of pHᵢ during growth, cells were stained with CFSE (Invitrogen) as suggested by the product manual and grown in MOPS media with 250 mg/L of each D-xylose, D-galactose, L-arabinose, and glycine. Several carbon sources were used to prevent favoring the growth of a subset of mutants, while at the same time allowing for full induction of the plasmid-borne rpoA. Variability introduced by the choice of carbon sources or other details in the protocol was accounted for by normalization. Media was withdrawn at different time points from each library and control cultures, put on ice and measured by flow cytometry (using a BD FACScan). The pHᵢ was calculated as the ratio of 585 to 530 nm emission when excited at 488 nm (Spilimbergo et al. 2005). Each time point was considered an entry in the distance vector for quantification of divergence. Two more entries of the distance vector were composed of pHᵢ values in non-growing cells. These were stained with BCECF-AM (Invitrogen), and resuspended in 10 mM phosphate buffer at either pH 5.0 or 7.0 immediately before FACS analysis (pHᵢ with this probe was calculated as the ratio of 650 to 530 nm emission when excited at 488 nm, as per manual recommendations). A sub-sample of 1500 data points was taken at random from each library and control data sets, and this sub-set was used to calculate the divergence as before (Klein-Marcuschamer and Stephanopoulos 2008); the algorithm was run 50 times and the divergence was averaged to smooth out the effects of sub-sampling. The exact values of the divergence varied somewhat with changes in the protocol, but the trends observed in FIG. 10 were maintained.

### Library Selection in Butyrate and Growth Assays

MOPS medium with 15 g/L butyrate was used for both selection and growth assays (initial pH adjusted to 7.0 with 6N HCl), except when trying the conditions described in FIG. 11. For selection, 30 mL of media were inoculated and cells were grown for about 20-24 hr, then a sample was transferred to a fresh batch of media. This procedure was repeated thrice, after which cells were spread in solid media overnight and individual colonies were picked for further study. Clones #1 and #16 in αCTD*L were chosen for their faster growth in butyrate, and their plasmids were purified and re-transformed into a clean K12 recA⁻ background to confirm the phenotype (FIG. 12). For growth assays, cells were cultured overnight in 15 g/L butyrate to avoid adaptation-related noise in the measurements and then diluted in the same media to obtain their growth curves. The mutant genes from clones #1 and #16 and the wild-type rpoA were transferred to a pCL1920 plasmid (which has the same origin of replication than pHACm, but confers streptomycin resistance, (Lerner and Inouye 1990)) and expressed from the P_{spc} promoter (Post et al. 1978).

### Divergence for Project Management

It is instructive to outline how the information given by measuring divergence can be used to guide a random strain improvement program. Assuming the perspective of someone responsible for managing an R&D project, we proposed the following strategy. Suppose a project with total budget T will be implemented to find an improved strain for a certain phenotype (all “costs” can be in units of money or time). A “random approach for finding an improved mutant” can be regarded as an iteration of two steps: building a library and quantifying its diversity (with cost B) and screening the library (with cost S). Initially, one builds and screens the library, incurring a cost B+S and leaving T−(B+S) for future experiments. If an improved mutant with characteristics above a certain threshold is isolated, the payoff to the R&D project is Y. Considering that the diversity metric of library i is a relative measure of the probability of finding an improved mutant (Klein-Marcuschamer and Stephanopoulos 2008), denoted Pᵢ, then the expected payoff can be written in terms of PᵢY. If no improved mutant is isolated in library 1, and T−(B+S)>B+S, then a second library can be constructed and screened. However, if after quantification of the diversity, it is observed that P₂<P₁, then the expected payoff is less for screening library 2 than library 1 (the associated risk is higher), and incurring a cost S>>B is not a good strategy.

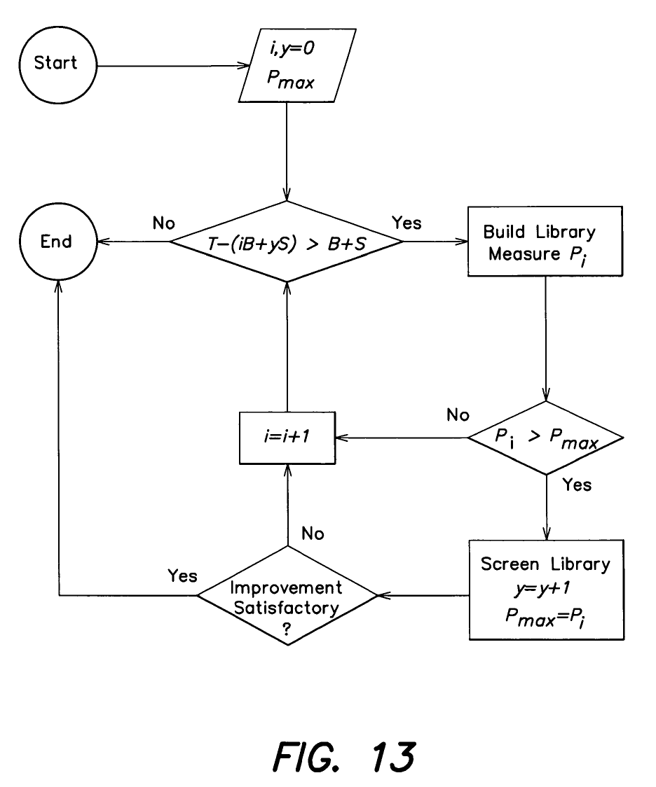

Now the budget is T−(2B+S); if this quantity is larger than B+S, then we can build a new library such that P₃>P₂. This can either be a library with similar characteristics to those of library 1, or preferentially, a library constructed with knowledge derived from the fact that we have established that P₁>P₂. Ideally, the new library is such that P₃>P₁>P₂. The process continues until the remaining budget is less than B+S or a variant with characteristics above the expected threshold is isolated. FIG. 13 outlines this process.

Stated differently: after each iteration, one can opt to continue or abandon the current approach for constructing libraries, and to continue or abandon the project altogether. Because screening is the resource- and labor-intensive step, it makes sense to carry it out only if the expected outcome of the experiment is better than that of constructing a new library, that is, if the a priori probability of finding a good mutant is larger than it was in the previous iteration. This process can continue until constructing new libraries becomes expensive or no obvious way of improving the library is available (e.g. by changing the mutation frequency, the targeting of mutations, etc.). In other words, evaluating libraries prior to screening them allows operational uncertainty to be resolved before expenses are incurred; therefore, the flexibility to abandon the approach has a concrete value (Huchzermeier and Loch 2001).

### Posterior Probability of Finding the Mutant in Different Libraries

We analyzed the probability of finding the S299T mutant (the posterior probability) in the different libraries that we constructed, using information about the length of the fragment that was subjected to mutagenesis, the average mutation frequency of each library, and assuming that the mutations follow a Poisson distribution (Firth and Patrick 2005). Table 2 shows that the S99T mutant could be found most frequently in the αCTD*L library, more than an order of magnitude more frequently than in any other library tested (this is the frequency of amplified PCR products at the DNA level, not the frequency in the cell library). Again, the population with the highest phenotypic diversity had the highest probability for the improved mutant to be found, which implies that we did not find the mutant in the αCTD*L library accidentally.

TABLE 2
Comparison of probabilities of finding the S299T mutant in different libraries

| | | Probability | Probability | Probability | |
|---|---|---|---|---|---|
| | Bases | of having 1 | of having the | of the change | Frequency |
| | subject to | mutation | mutation in | being the one | of mutant |
| | mutagenesis | occurring | the right base | required | (one in:) |
| rpoA*L | 1300 | 7.33E−02 | 7.69E−04 | 0.33 | 5.32E+04 |
| rpoA*M | 1300 | 6.38E−03 | 7.69E−04 | 0.33 | 6.11E+05 |
| rpoA*H | 1300 | 1.11E−03 | 7.69E−04 | 0.33 | 3.51E+06 |
| aCTD*L | 250 | 3.58E−01 | 4.00E−03 | 0.33 | 2.09E+03 |
| aCTD*H | 250 | 1.49E−02 | 4.00E−03 | 0.33 | 5.04E+04 |

### REFERENCES

Alper, H., J. Moxley, E. Nevoigt, G. R. Fink, and G. Stephanopoulos. 2006. Engineering yeast transcription machinery for improved ethanol tolerance and production. Science 314:1565-8.
Alper, H., and G. Stephanopoulos. 2007. Global transcription machinery engineering: A new approach for improving cellular phenotype. Metab Eng 9:258-67.
Aukrust, T. W., M. B. Brurberg, and I. F. Nes. 1995. Transformation of Lactobacillus by electroporation. Methods Mol Biol 47:201-8.
Azcarate-Peril, M. A., E. Alternann, R. L. Hoover-Fitzula, R. J. Cano, and T. R. Klaenhammer. 2004. Identification and inactivation of genetic loci involved with Lactobacillus acidophilus acid tolerance. Appl Environ Microbiol 70:5315-22.
Bellemann P, Bereswill S, Berger S, Geider K. 1994. Visualization of capsule formation by Erwinia amylovora and assays to determine amylovoran synthesis. Int J Biol Macromol 16 (6): 290-296.
Beltran, A., Y. Liu, S. Parikh, B. Temple, and P. Blancafort. 2006. Interrogating genomes with combinatorial artificial transcription factor libraries: asking zinc finger questions. Assay Drug Dev Technol 4:317-31.
Bitter T, Muir M. 1962. A modified uronic acid carbazole reaction. Anal Biochem 4: 330-334.
Bonomo, J., M. D. Lynch, T. Warnecke, J. V. Price, and R. T. Gill. 2008. Genome-scale analysis of anti-metabolite directed strain engineering. Metab Eng 10:109-20.
Booth, I. R. 1985. Regulation of cytoplasmic pH in bacteria. Microbiol. Rev 49:359-78.
Busby S, Ebright R H. 1994. Promoter structure, promoter recognition, and transcription activation in prokaryotes. Cell 79: 743-46.
Campbell, E. A., O. Muzzin, M. Chlenov, J. L. Sun, C. A. Olson, O. Weinman, M. L. Trester-Zedlitz, and S. A. Darst. 2002. Structure of the bacterial RNA polymerase promoter specificity sigma subunit. Mol Cell 9:527-39.
Chen H, Tang H, Ebright R H. 2003. Functional interaction between RNA polymerase a aubunit C-terminal domain and s70 in UP-Element- and activator-dependent transcription. Mol Cell 11: 1621-33.
Chong B F, Nielsen L K. 2003. Amplifying the cellular reduction potential of Streptococcus zooepidemicus. J Biotechnol 100: 33-41.
Day, D. A., and M. F. Tuite. 1998. Post-transcriptional gene regulatory mechanisms in eukaryotes: an overview. J Endocrinol 157:361-71.
Demain, A. L., J. E. Davies, and R. M. Atlas. 1999. Manual of industrial microbiology and biotechnology, 2nd ed. ASM Press, Washington, D.C.
Dombroski, A. J., W. A. Walter, M. T. Record, Jr., D. A. Siegele, and C. A. Gross. 1992. Polypeptides containing highly conserved regions of transcription initiation factor sigma 70 exhibit specificity of binding to promoter DNA. Cell 70:501-12.
Duy, N. V., U. Mader, N. P. Tran, J. F. Cavin, T. Tam le, D. Albrecht, M. Hecker, and H. Antelmann. 2007. The proteome and transcriptome analysis of Bacillus subtilis in response to salicylic acid. Proteomics 7:698-710.
Elowitz, M. B., A. J. Levine, E. D. Siggia, and P. S. Swain. 2002. Stochastic gene expression in a single cell. Science 297:1183-6.
Errington, J. 1991. Possible intermediate steps in the evolution of a prokaryotic developmental system. Proc Biol Sci 244:117-21.
Firth, A. E., and W. M. Patrick. 2005. Statistics of protein library construction. Bioinformatics 21:3314-5.
Fischer, C. R., and A. Peterson. 22 Feb. 2008. Conversion of natural products including cellulose to hydrocarbons, hydrogen and/or related compounds. US patent PCT/US2008/002412; published on Aug. 28, 2008 as WO2008/103480.
Follstad B, Balcarcel R, Wang D I C, Stephanopoulos G. 1999. Metabolic flux analysis of hybridoma continuous culture steady state multiplicity. Biotechnol Bioeng 63: 675-683.
Franck P, et al. 1996. Measurement of intracellular pH in cultured cells by flow cytometry with BCECF-AM. J Biotechnol 46: 187-95.
Gaal, T., W. Ross, E. E. Blatter, H. Tang, X. Jia, V. V. Krishnan, N. Assa-Munt, R. H. Ebright, and R. L. Gourse. 1996. DNA-binding determinants of the alpha subunit of RNA polymerase: novel DNA-binding domain architecture. Genes Dev 10:16-26.
Giraud, E., B. Lelong, and M. Raimbault. 1991. Influence of Ph and Initial Lactate Concentration on the Growth of Lactobacillus-Plantarum. Applied Microbiology and Biotechnology 36:96-99.
Goa K L, Benfield P. 1994. Hyaluronic acid: a review of its pharmacology and use as a surgical aid in opthalmology and its therapeutic potential in joint disease and wound healing. Drugs 47: 536-566.
Gregory B D, Nickels B E, Darst S A. 2005. An altered-specificity DNA-binding mutant of Escherichia coli σ70 facilitates the analysis of σ70 function in vivo. Mol Microbiol 56 (5): 1208-1219.
Hansen, M. E., F. Lund, and J. M. Carstensen. 2003. Visual clone identification of Penicillium commune isolates. J Microbiol Methods 52:221-9.
Helmann J D, Chamberlin M J. 1988. Structure and function of bacterial sigma factors. Ann Rev Biochem 57: 839-72.
Hoover D M, Lubkowski J. 2002. DNAWorks: an automated method for designing oligonucleotides for PCR-based gene synthesis. Nucleic Acids Res 30(10): e43.
Huchzermeier, A., and C. H. Loch. 2001. Project management under risk: Using the real options approach to evaluate flexibility in R&D. Management Science 47:85-101.
Imashimizu, M., M. Hanaoka, A. Seki, K. S. Murakami, and K. Tanaka. 2006. The cyanobacterial principal sigma factor region 1.1 is involved in DNA-binding in the free form and in transcription activity as holoenzyme. FEBS Lett 580:3439-44.
Ishihama A. 1992. Role of the RNA polymerase alpha subunit in transcription activation. Mol Microbiol 6 :3283-88.
Jeon, Y. H., T. Negishi, M. Shirakawa, T. Yamazaki, N. Fujita, A. Ishihama, and Y. Kyogoku. 1995. Solution structure of the activator contact domain of the RNA polymerase alpha subunit. Science 270:1495-7.
Jin Y S, Alper H, Yang Y T, Stephanopoulos G. 2005. Improvement of xylose uptake and ethanol production in recombinant Saccharomyces cerevisiae through inverse metabolic engineering approach. Appl Env Microb 71 (12): 8249-8256.
Jin, Y. S., and G. Stephanopoulos. 2007. Multi-dimensional gene target search for improving lycopene biosynthesis in Escherichia coli. Metab Eng 9:337-47.
Kakizaki I, Takagaki K, Endo Y, Kudo D, Ikeya H, Miyoshi T, Baggenstoss B A, Tlapak-Simmons V L, Kumari K, Nakane A, Weigel P H, Endo M. 2002. Inhibition of hyaluronan synthesis in Streptococcus equi FM100 by 4-methylumbelliferone. Eur J Biochem 269: 5066-5075.
Kim J H, Yoo S J, Oh D K, Kweon Y G, Park D W, Lee C H, Gil G H. 1996. Selection of a Streptococcus equi mutant and optimization of culture conditions for the production of high molecular weight hyaluronic acid. Enz Microb Technol 19: 440-445.
Kiss R D, Stephanopoulos G. 1991. Metabolic activity control of L-lysine fermentation by restrained growth fed-batch strategies. Biotechnol Prog 7: 501-509.
Kimura, M., and A. Ishihama. 1995. Functional map of the alpha subunit of Escherichia coli RNA polymerase: insertion analysis of the amino-terminal assembly domain. J Mol Biol 248:756-67.
Kitten, J., B. Borup, R. Voladari, and K. Zahn. 2005. Parallel capillary electrophoresis for the quantitative screening of fermentation broths containing natural products. Metab Eng 7:53-8.
Kleerebezem, M., J. Boekhorst, R. van Kranenburg, D. Molenaar, O. P. Kuipers, R. Leer, R. Tarchini, S. A. Peters, H. M. Sandbrink, M. W. Fiers, W. Stiekema, R. M. Lankhorst, P. A. Bron, S. M. Hoffer, M. N. Groot, R. Kerkhoven, M. de Vries, B. Ursing, W. M. de Vos, and R. J. Siezen. 2003. Complete genome sequence of Lactobacillus plantarum WCFS1. Proc Natl Acad Sci USA 100:1990-5.
Klein-Marcuschamer, D., C. N. S. Santos, H. Yu, and G. Stephanopoulos. 2008. Mutagenesis of the bacterial RNA polymerase alpha subunit for improving complex phenotypes. Applied and Environmental Microbiology, submitted.
Klein-Marcuschamer, D., and G. Stephanopoulos. 2008. Assessing the potential of mutational strategies to elicit new phenotypes in industrial strains. Proc Natl Acad Sci USA 105:2319-24.
Kok, J., J. M. van der Vossen, and G. Venema. 1984. Construction of plasmid cloning vectors for lactic streptococci which also replicate in Bacillus subtilis and Escherichia coli. Appl Environ Microbiol 48:726-31.
Kresnowati, M. T., C. Suarez-Mendez, M. K. Groothuizen, W. A. van Winden, and J. J. Heijnen. 2007. Measurement of fast dynamic intracellular pH in Saccharomyces cerevisiae using benzoic acid pulse. Biotechnol Bioeng 97:86-98.
Kresnowati, M. T., C. M. Suarez-Mendez, W. A. van Winden, W. M. van Gulik, and J. J. Heijnen. 2008. Quantitative physiological study of the fast dynamics in the intracellular pH of Saccharomyces cerevisiae in response to glucose and ethanol pulses. Metab Eng 10:39-54.
Lauren T C. 1998. The chemistry, biology and medical applications of hyaluronan and its derivatives. Portland Press Ltd, London.
Lerner, C. G., and M. Inouye. 1990. Low copy number plasmids for regulated low-level expression of cloned genes in Escherichia coli with blue/white insert screening capability. Nucleic Acids Res 18:4631.
Lynd, L. R., C. E. Wyman, and T. U. Gerngross. 1999. Biocommodity Engineering. Biotechnol Prog 15:777-793.
Martinez-Antonio, A., S. C. Janga, and D. Thieffry. 2008. Functional organisation of Escherichia coli transcriptional regulatory network. J Mol Biol 381:238-47.
McDonald, L. C., H. P. Fleming, and H. M. Hassan. 1990. Acid Tolerance of Leuconostoc mesenteroides and Lactobacillus plantarum. Appl Environ Microbiol 56:2120-2124.
Miller, J. H. 1972. Experiments in molecular genetics, p. 125-129. Cold Spring Harbor Laboratory, Cold Spring Harbor, N.Y.
Miyagishi, M., S. Matsumoto, H. Akashi, H. Kawasaki, T. Fukao, Y. Fukuda, M. Sano, Y. Kato, Y. Takagi, Y. Tanaka, M. Warashina, T. Kuwabara, S. Y. Sawata, Y. Ikeda, S. Kawahara, K. C. Sunil, R. Wadhwa, and K. Taira. 2005. Chemistry-based RNA technologies: demonstration of usefulness of libraries of ribozymes and short hairpin RNAs (shRNAs). Nucleic Acids Symp Ser (Oxf):91-2.
Murakami, K., N. Fujita, and A. Ishihama. 1996. Transcription factor recognition surface on the RNA polymerase alpha subunit is involved in contact with the DNA enhancer element. Embo J 15:4358-67.
Murphy, M. G., L. O'Connor, D. Walsh, and S. Condon. 1985. Oxygen dependent lactate utilization by Lactobacillus plantarum. Arch Microbiol 141:75-9.
Neidhardt F C, Bloch P L, Smith D F. 1974. Culture medium for Enterobacteria. J Bacteriol 119 (3): 736-747.
Niu, W., Y. Kim, G. Tau, T. Heyduk, and R. H. Ebright. 1996. Transcription activation at class II CAP-dependent promoters: two interactions between CAP and RNA polymerase. Cell 87:1123-34.
Ogrodowski C S, Hokka C O, Santana M H A. 2005. Production of hyaluronic acid by Streptococcus. Appl Biochem Biotechnol 121-124: 753-761.
Park, K. S., D. K. Lee, H. Lee, Y. Lee, Y. S. Jang, Y. H. Kim, H. Y. Yang, S. I. Lee, W. Seol, and J. S. Kim. 2003. Phenotypic alteration of eukaryotic cells using randomized libraries of artificial transcription factors. Nat Biotechnol 21:1208-14.
Park, K. S., Y. S. Jang, H. Lee, and J. S. Kim. 2005a. Phenotypic alteration and target gene identification using combinatorial libraries of zinc finger proteins in prokaryotic cells. Bacteriol 187:5496-9.
Park, K. S., W. Seol, H. Y. Yang, S. I. Lee, S. K. Kim, R. J. Kwon, E. J. Kim, Y. H. Roh, B. L. Seong, and J. S. Kim. 2005b. Identification and use of zinc finger transcription factors that increase production of recombinant proteins in yeast and mammalian cells. Biotechnol Prog 21:664-70.
Patnaik, R., S. Louie, V. Gavrilovic, K. Perry, W. P. Stemmer, C. M. Ryan, and S. del Cardayre. 2002. Genome shuffling of Lactobacillus for improved acid tolerance. Nat Biotechnol 20:707-12.
Penney D P, Powers J M, Frank M, Willis C, Churukian C. 2002. Analysis and testing of biological stains: The biological stain commission procedures. Biotechnic Histochem 77 (5&6): 237-275.
Pieterse, B., R. J. Leer, F. H. Schuren, and M. J. van der Werf. 2005. Unravelling the multiple effects of lactic acid stress on Lactobacillus plantarum by transcription profiling. Microbiology 151:3881-94.
Porro, D., M. M. Bianchi, L. Brambilla, R. Menghini, D. Bolzani, V. Carrera, J. Lievense, C. L. Liu, B. M. Ranzi, L. Frontali, and L. Alberghina. 1999. Replacement of a metabolic pathway for large-scale production of lactic acid from engineered yeasts. Appl Environ Microbiol 65:4211-5.
Posno, M., R. J. Leer, N. van Luijk, M. J. van Giezen, P. T. Heuvelmans, B. C. Lokman, and P. H. Pouwels. 1991. Incompatibility of Lactobacillus Vectors with Replicons Derived from Small Cryptic Lactobacillus Plasmids and Segregational Instability of the Introduced Vectors. Appl Environ Microbiol 57:1822-1828.
Post, L. E., A. E. Arfsten, F. Reusser, and M. Nomura. 1978. DNA sequences of promoter regions for the str and spc ribosomal protein operons in E. coli. Cell 15:215-29.
Ross, W., and R. L. Gourse. 2005. Sequence-independent upstream DNA-alphαCTD interactions strongly stimulate Escherichia coli RNA polymerase-lacUV5 promoter association. Proc Natl Acad Sci USA 102:291-6.
Rowlands, R. T. 1984. Industrial Strain Improvement—Mutagenesis and Random Screening Procedures. Enzyme and Microbial Technology 6:3-10.
Sambrook J, Fritsch E F, and Maniatis T. 1988. Molecular cloning: a laboratory manual, 2nd ed. Cold Spring Harbor Laboratory Press, Cold Spring Harbor, N.Y.
San K Y, Stephanopoulos G. 1984. Studies on on-line bioreactor identification. IV. Utilization of pH measurements for product estimation. Biotech Bioeng 26: 1209-1218.
Santos, C. N., and G. Stephanopoulos. 2008. Combinatorial engineering of microbes for optimizing cellular phenotype. Curr Opin Chem Biol 12:168-76.
Spilimbergo, S., A. Bertucco, G. Basso, and G. Bertoloni. 2005. Determination of extracellular and intracellular pH of Bacillus subtilis suspension under CO2 treatment. Biotechnol Bioeng 92:447-51.
Stephanopoulos G, Fredrickson A G, Aris R. 1979. The growth of competing microbial populations in a CSTR with periodically varying inputs. AIChE J 25: 863-872.
Stephanopoulos G, Sinskey A J. 1993. Metabolic engineering: issues and methodologies. Trends in Biotechnol 11: 392-396.
Stephanopoulos G, Simpson T W. 1997. Flux amplification in complex metabolic networks. Chem Eng Sci 52: 2607-2627.
Stephanopoulos G, Kelleher J. 2001. How to make a superior cell. Science 292: 2024-2026.
Stephanopoulos, G. 2002. Metabolic engineering by genome shuffling. Nat Biotechnol 20:666-8.
Stephanopoulos, G., H. Alper, and J. Moxley. 2004. Exploiting biological complexity for strain improvement through systems biology. Nat Biotechnol 22:1261-7.
Stutzman-Engwall, K., S. Conlon, R. Fedechko, H. McArthur, K. Pekrun, Y. Chen, S. Jenne, C. La, N. Trinh, S. Kim, Y. X. Zhang, R. Fox, C. Gustafsson, and A. Krebber. 2005. Semi-synthetic DNA shuffling of aveC leads to improved industrial scale production of doramectin by Streptomyces avermitilis. Metab Eng 7:27-37.
Swain, P. S., M. B. Elowitz, and E. D. Siggia. 2002. Intrinsic and extrinsic contributions to stochasticity in gene expression. Proc Natl Acad Sci USA 99:12795-800.
Tashiro, Y., K. Takeda, G. Kobayashi, K. Sonomoto, A. Ishizaki, and S. Yoshino. 2004. High butanol production by Clostridium saccharoperbutylacetonicum N1-4 in fed-batch culture with pH-Stat continuous butyric acid and glucose feeding method. J Biosci Bioeng 98:263-8.
Vallino J J, Stephanopoulos G. 1994. Carbon flux distributions at the glucose-6 phosphate branch point in Corynebacterium glutamicum during lysine overproduction. Biotechnol Prog 10: 320-326.
Venturi V. 2003. Control of rpoS transcription in Escherichia coli and Pseudomonas: why so different? Mol Microb 49(1): 1-9.
Wang F L, Lee S Y. 1998. High cell density culture of metabolically engineered Escherichia coli for the production of poly (3-hydroxybutyrate) in a defined medium. Biotechnol Bioeng 58 (2&3): 325-328.
Warnecke, T. E., M. D. Lynch, A. Karimpour-Fard, N. Sandoval, and R. T. Gill. 2008. A genomics approach to improve the analysis and design of strain selections. Metab Eng 10:154-65.
Widner B R, Behr S, Dollen V, Tang M, Heu T, Sloma A, Sternberg D, DeAngelis P L, Weigel P H, Brown S. 2005. Hyaluronic acid production in Bacillus subtilis. Appl Environ Microbiol 71 (7): 3747-3752.
Yu H M, Stephanopoulos G. 2008. Metabolic engineering of Escherichia coli for biosynthesis of hyaluronic acid. Metab Eng. 10(1):24-32.
Yu, H., K. Tyo, H. Alper, D. Klein-Marcuschamer, and G. Stephanopoulos. 2008. A high-throughput screen for hyaluronic acid accumulation in recombinant Escherichia coli transformed by libraries of engineered sigma factors. Biotechnol Bioeng. 101(4):788-96.
Zhang, Y. X., K. Perry, V. A. Vinci, K. Powell, W. P. Stemmer, and S. B. del Cardayre. 2002. Genome shuffling leads to rapid phenotypic improvement in bacteria. Nature 415:644-6.
Zigova, J., and E. Sturdik. 2000. Advances in biotechnological production of butyric acid. Journal of Industrial Microbiology & Biotechnology 24:153-160.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety for the purposes disclosed above.

We claim:

1. A method for altering the phenotype of a cell comprising:
mutating a nucleic acid encoding ribonucleic acid polymerase (RNAP) alpha subunit RpoA and, optionally, its promoter,
expressing the nucleic acid in a prokaryotic cell to provide an altered cell that includes the mutated nucleic acid encoding RpoA, and
culturing the altered cell.

2. The method of claim 1, further comprising determining the phenotype of the altered cell.

3. The method of claim 1, further comprising repeating the mutation of the nucleic acid to produce a n^{th} generation altered cell.

4.-9. (canceled)

10. The method of claim 1, wherein the nucleic acid is part of an expression vector.

11. The method of claim 1, wherein the nucleic acid is a member of a collection of nucleic acids.

12. (canceled)

13. The method of claim 1, wherein the step of expressing the nucleic acid comprises integrating the nucleic acid into the genome or replacing a nucleic acid that encodes the endogenous RpoA.

14. The method of claim 1, wherein the mutation of the nucleic acid comprises directed evolution of the nucleic acid.

15. (canceled)

16. (canceled)

17. The method of claim 1, wherein the mutation of the nucleic acid comprises synthesizing the nucleic acid with one or more mutations.

18.-27. (canceled)

28. The method of claim 1, further comprising selecting the altered cell for a predetermined phenotype.

29. (canceled)

30. (canceled)

31. The method of claim 1, wherein the phenotype is increased tolerance of deleterious culture conditions.

32.-40. (canceled)

41. The method of claim 1, wherein the phenotype is increased metabolite production.

42.-45. (canceled)

46. The method of claim 1, wherein the phenotype is tolerance to a toxic substrate, metabolic intermediate or product.

47.-49. (canceled)

50. The method of claim 1, wherein the phenotype is antibiotic resistance.

51. The method of claim 1, wherein the cell used in the method is optimized for the phenotype prior to mutating the nucleic acid encoding RpoA.

52. The method of claim 1, further comprising identifying the changes in gene expression in the altered cell.

53. (canceled)

54. A method for altering the phenotype of a cell comprising altering the expression of one or more gene products in a first cell that are identified by detecting changes in gene expression in a second cell, wherein the changes in gene expression in the second cell are produced by mutating a nucleic acid encoding ribonucleic acid polymerase (RNAP) alpha subunit RpoA of the second cell.

55. The method of claim 54, wherein altering the expression of the one or more gene products in the first cell comprises increasing expression of one or more gene products that were increased in the second cell.

56.-65. (canceled)

66. A method for altering the production of a metabolite, comprising
mutating, according to claim 1, ribonucleic acid polymerase (RNAP) alpha subunit RpoA of a prokaryotic cell that produces a selected metabolite to produce an altered cell, and
isolating altered cells that produce increased or decreased amounts of the selected metabolite.

67. The method of claim 66, wherein the method further comprises
culturing the isolated cells, and
recovering the metabolite from the cells or the cell culture.

68.-73. (canceled)

74. A collection comprising a plurality of different nucleic acid molecule species, wherein each nucleic acid molecule species encodes ribonucleic acid polymerase (RNAP) alpha subunit RpoA comprising different mutation(s).

75. (canceled)

76. (canceled)

77. The collection of claim 74, wherein the nucleic acid molecule species are contained in expression vectors.

78. The collection of claim 77, wherein the expression vectors contain a plurality of different nucleic acid molecule species, wherein each nucleic acid molecule species encodes different RNAP alpha subunit RpoA mutations.

79. The collection of claim 74, wherein the nucleic acid encoding RpoA is mutated by directed evolution.

80.-88. (canceled)

89. A collection of cells comprising the collection of nucleic acid molecules of claim 74.

90. The collection of claim 89, comprising a plurality of cells, each of the plurality of cells comprising one or more of the nucleic acid molecules.

91.-120. (canceled)

121. A method of producing a cell that is tolerant to butyrate, the method comprising:
mutating the alpha CTD domain of a nucleic acid encoding ribonucleic acid polymerase (RNAP) alpha subunit RpoA,
expressing the nucleic acid in a cell to provide an altered cell that includes the mutated nucleic acid encoding RpoA,
culturing the cell in butyrate, and
isolating a cell that is tolerant to butyrate.

122. The method of claim 121 wherein the mutation in RNAP is a substitution of amino acid 299, optionally from a serine residue to a threonine residue.

123.-130. (canceled)


SEQUENCE LISTING (11 sequences)

SEQ ID NO: 1 - DNA, 37 bp, Artificial Sequence
  Oligonucleotide primer
gcgcgcccgggacgttgtaagcattcgtgagaaagcg

SEQ ID NO: 2 - DNA, 37 bp, Artificial Sequence
  Oligonucleotide primer
gcgcggtgcactggcgcatgaccttatcctctcagta

SEQ ID NO: 3 - DNA, 41 bp, Artificial Sequence
  Oligonucleotide primer
aacctaggagctctgatttaacggcttaagtgccgaagagc

SEQ ID NO: 4 - DNA, 37 bp, Artificial Sequence
  Oligonucleotide primer
tggaagctttaacgcctgatccggcctaccgattaat

SEQ ID NO: 5 - DNA, 41 bp, Artificial Sequence
  Oligonucleotide primer
aacctaggagctcagactggcctttctgacagatgcttact

SEQ ID NO: 6 - DNA, 41 bp, Artificial Sequence; identical to SEQ ID NO: 5
  Oligonucleotide primer

SEQ ID NO: 7 - DNA, 37 bp, Artificial Sequence; identical to SEQ ID NO: 1
  Oligonucleotide primer

SEQ ID NO: 8 - DNA, 38 bp, Artificial Sequence
  Oligonucleotide primer
gcgcggtgcactggcgcatgaccttatccttctcagta

SEQ ID NO: 9 - DNA, 39 bp, Artificial Sequence
  Oligonucleotide primer
acgtgacgtacgtcagcctgaagtgaaagaagagaaacc

SEQ ID NO: 10 - DNA, 100 bp, Artificial Sequence
  Oligonucleotide primer
tatcggagatctggtacagcgtaccgaggttgagctccttaaaacgcctaaccttggtaaaaaatctcttactgagattaaagacgtgctggcttcccgt

SEQ ID NO: 11 - DNA, 100 bp, Artificial Sequence
  Oligonucleotide primer
tgtaccagatctccgatatagtggatacgttctgctttaaggcagttagcagagcggacagtcaattccagatcgtcaacagggcgcagcaggatcggat